(19) United States
(12) Patent Application Publication
(10) Pub. No.: US 2022/0175827 A1
(43) Pub. Date: Jun. 9, 2022

(54) Biosynthesis of Selenium Nanoparticles Having Antimicrobial Activity

(71) Applicant: Northeastern University, Boston, MA (US)

(72) Inventors: David Medina CRUZ, Boston, MA (US); Thomas J. WEBSTER, Barrington, RI (US)

(21) Appl. No.: 17/600,652

(22) PCT Filed: Apr. 6, 2020
(86) PCT No.: PCT/US2020/026947

§ 371 Date: Oct. 1, 2021

Related U.S. Application Data
(60) Provisional application No. 62/830,344, filed on Apr. 5, 2019.

(51) Int. Cl. A61K 33/04; A61K 9/51; A61K 51/12; A61P 31/04

(52) U.S. Cl. CPC A61K 33/04; A61K 9/51; A61P 31/04; A61K 51/1244; A61K 9/5192

(57) ABSTRACT

Selenium (Se) nanostructures are synthesized using bacteria, and the synthetic method provides options for specific functionalization of the nanostructures, targeting, as well as options for crystal form of and for additives to the composition. In addition to drug delivery and imaging options, the synthesized Se nanostructures provide methods of inhibiting drug resistant bacterial cells and cancer cells without cytotoxicity towards normal human cells and dermal fibroblasts. The green chemistry methods for synthesizing Se nanostructures do not produce toxic byproducts and do not require toxic reagents in comparison to traditional chemical synthetic methods for making Se nanostructures, while simultaneously producing new therapeutic benefits and treatments.

### CROSS-REFERENCE TO RELATED APPLICATIONS

This application claims priority to U.S. Provisional Application No. 62/830,344, filed 5 Apr. 2019, the entirety of which is incorporated herein by reference.

### BACKGROUND

Green nanotechnology was born as the application of the twelve principles of green chemistry to nanotechnology gave rise to more efficient, environmentally-friendly, cost-effective, and responsible synthesis and use of nanomaterials. The application of nanomaterials to medicine brought the use of green nanotechnology to medicine, with various nanostructures being used as powerful agents towards bacterial infections, cancer, and in other areas such as drug delivery, imaging, and biosensors.

Integration of green nanotechnology into medicine has led to better environmental outcomes while providing insight into bio-adaptable synthetic processes and alternative treatments for complex and resistant diseases. In one aspect, green nanotechnology provides new treatments for the most complex ailments as the same technology expands to provide responsible alternatives to global environmental change. Thus, taking the health of the environment into account leads to new synthetic methods integrating bio-machinery to produce less waste, use less chemicals, use less energy, while more efficiently producing products, and new treatments are provided by the same principles. Green nanotechnology provides new data, new understanding, and new adaptations as the mechanisms provided in one area of green nanotechnology expand to new frontiers.

Bacteria have been used to generate nanomaterials in an environmentally friendly manner. Synthesis of nanoparticles has been proposed using bacterial cells and extracts.

Elemental selenium has been found to have many effects in living systems as well as useful optical and physical properties. Selenium has several allotropes that can change depending a rate of temperature change. For example, in one amorphous form, selenium can appear red, in a vitreous form, or black, and different crystal forms can be obtained depending on conditions. While the biological role of selenium has been under investigation for many years, much remains to be learned about its role in biological systems. Plants that accumulate selenium from the environment have been tied to detrimental biological effects. While beneficial and therapeutic effects of selenium have been identified, they can depend on the size of nanoparticles, crystal form, salt form, functionalization, specificity, and dose. Selenium nanoparticles have gained attention for their capability to inhibit the growth of bacteria and for their ability to treat cancer.

Further refinement of selenium nanotechnology is needed to control the synthesis, form, and selectivity of selenium nanoparticles.

### SUMMARY

Selenium nanoparticles can be synthesized through the use of bacteria to carry out selenium reduction. The selenium nanoparticles (SeNPs) possess antibacterial activity toward both Gram-negative and Gram-positive bacteria, as well as drug-resistant forms, but show no significant cytotoxicity toward fibroblasts at antibacterial concentrations. The SeNPs also possess an anticancer effect, causing a consistent delay in melanoma cell growth over trace nanoparticle concentrations to 100 μg/mL.

The technology described herein provides methods for making tunable and targeted selenium nanoparticles (SeNPs) with different contents of crystal forms and various coatings, depending on the bacteria used for synthesis and the synthetic conditions. The SeNPs can have a partial or complete coating. The coating can provide specificity for targeted therapies.

The present technology can be further summarized by the following features.

1. A method of inhibiting the growth of a drug-resistant bacterial pathogen in a subject, the method comprising administering selenium nanoparticles to the subject, whereby the growth of the bacterial pathogen in the subject is inhibited;

wherein the selenium nanoparticles are produced by a process comprising growing the bacterial pathogen in the presence of a selenium salt, whereby selenium ions of the selenium salt are reduced to elemental selenium to form the selenium nanoparticles; and

wherein the selenium nanoparticles selectively inhibit growth of the drug-resistant bacterial pathogen compared to inhibition by the selenium nanoparticles of growth of a non-drug-resistant form of the bacterial pathogen.

2. The method of feature 1, wherein the selenium nanoparticles are at least partially coated with organic molecules provided by the bacterial pathogen during the process of producing the selenium nanoparticles.
3. The method of feature 1 or 2, wherein the drug-resistant bacterial pathogen is of the same species as the non-drug-resistant form of the bacterial pathogen.
4. The method of any of the preceding features, wherein both the drug-resistant and non-drug-resistant forms of the bacterial pathogen are Escherichia coli, or both the drug-resistant and non-drug-resistant forms are Staphylococcus aureus.
5. The method of any of the preceding features, wherein a minimum inhibitory concentration of the selenium nanoparticles for the drug-resistant bacterial pathogen is less than about 30 micrograms/mL.
6. The method of any of the preceding features, further comprising, prior to said administering:

collecting a sample of the drug-resistant bacterial pathogen from the subject;

cultivating the collected drug-resistant bacterial pathogen in vitro; and

forming said selenium nanoparticles by growing the cultivated bacterial pathogen in the presence of said selenium salt, whereby selenium ions of the selenium salt are reduced to elemental selenium to form said selenium nanoparticles.

7. The method of any of the preceding features, wherein the administered selenium nanoparticles are formulated with one or more pharmaceutically acceptable excipients.
8. The method of any of the preceding features, wherein the administered selenium nanoparticles comprise one or more radioisotopes, and the method further comprises performing radioimaging of the subject, irradiation of the subject by the selenium nanoparticles, or absorption of radiation from the selenium nanoparticles by elemental selenium in the nanoparticles and emission of energy from the selenium nanoparticles.
9. The method of any of the preceding features, wherein the selenium nanoparticles possess magnetic properties operative to collect, concentrate, organize, dissipate, or repel the nanoparticles.
10. The method of any of the preceding features, wherein the selenium nanoparticles comprise a moiety selected from the group consisting of a protein, an antibody, an oligonucleotide, and a small molecule drug.
11. The method of feature 10, wherein the moiety is a targeting moiety capable of targeting the selenium nanoparticles to the drug-resistant bacterial pathogen or to a cell of the subject.
12. The method of any of the preceding features, wherein the selenium nanoparticles cause a lethal increase in reactive oxygen species in the drug resistant bacteria.
13. A method of inhibiting the growth of cancer cells, the method comprising administering to a subject in need thereof a therapeutically effective amount of selenium nanoparticles; wherein the selenium nanoparticles are produced by a process comprising growing bacteria in the presence of a selenium salt wherein selenium ions of the salt are reduced to elemental selenium to form the nanoparticles.
14. The method of feature 13, wherein the cancer cells are cells of a cancer selected from the group consisting of skin cancer, lung cancer, breast cancer, prostate cancer, colorectal cancer, bladder cancer, melanoma, Non-Hodgkin lymphoma, kidney cancer, and leukemia.
15. The method of feature 13 or 14, wherein the growth of non-cancerous cells in the subject is not substantially inhibited.
16. The method of feature 15, wherein the therapeutically effective amount provides a concentration of selenium nanoparticles not greater than about 25 micrograms/mL at or near the cancer cells.
17. The method of any of features 13-16, wherein the selenium nanoparticles cause a lethal increase in reactive oxygen species in the cancer cells.
18. Selenium nanoparticles produced by a process comprising growing a first type of bacteria in the presence of a selenium salt, wherein selenium ions of the salt are reduced to elemental selenium, wherein the selenium nanoparticles selectively inhibit growth of the first type of bacteria more than the selenium nanoparticles inhibit growth of a second type of bacteria.
19. The selenium nanoparticles of feature 18, wherein the selenium nanoparticles are at least partially coated with organic molecules provided by the bacterial pathogen during the process of producing the selenium nanoparticle.
20. The selenium nanoparticles of feature 19, wherein the organic coating causes the selenium nanoparticles to selectively inhibit growth of the first type of bacteria compared to other types of bacteria.
21. The selenium nanoparticles of feature 19 or 20, wherein the organic coating comprises one or more proteins.
22. The selenium nanoparticles of any of features 18-21, wherein the selenium nanoparticles further comprise a moiety selected from the group consisting of a radioisotope, a protein, an antibody, an oligonucleotide, a small molecule, and a therapeutic agent.
23. The selenium nanoparticles of any of features 18-22, wherein the first type of bacteria is drug-resistant.
24. The selenium nanoparticles of feature 23, wherein the drug resistance is antibiotic resistance.
25. The selenium nanoparticles of any of features 18-24, wherein the first bacteria are multi-drug resistant Escherichia coli or methicillin-resistant Staphylococcus aureus.
28. The selenium nanoparticles of any of features 18-25, wherein the selenium nanoparticles comprise amorphous selenium and/or trigonal selenium crystal structure.
29. The selenium nanoparticles of any of features 18-28, wherein the nanoparticles have an average diameter in the range from about 50 nm to about 110 nm, or about 50 to about 75 nm, or about 70 nm to about 110 nm.
30. The selenium nanoparticles of any of features 18-29, wherein the organic coating is operative to stabilize the selenium nanoparticles as a colloid or suspension for at least about 60 days.
31. The selenium nanoparticles of any of features 18-30, wherein the organic coating provides a Z-potential value exceeding ±30 mV which is stable for at least about 60 days.
32. The selenium nanoparticles of any of features 18-31, wherein the first type of bacteria is a drug-resistant form of the second type of bacteria.
33. The selenium nanoparticles of any of features 18-32 that are capable of inhibiting proliferation of cancer cells without significantly inhibiting proliferation of non-cancer cells of a human subject.
34. The selenium nanoparticles of feature 33, wherein the cancer cells are melanoma cells and the normal cells are dermal fibroblasts.
35. A pharmaceutical composition comprising the selenium nanoparticles of any of features 18-34 and a pharmaceutically acceptable excipient.
36. A kit for inhibiting the growth of drug-resistant bacteria, the kit comprising

a selenium salt; and

instructions for carrying out the method of feature 6.

The green methods provided herein can be accomplished using minimal ingredients and can be quickly modified to adapt to treating various drug resistant bacteria and infections, for example, in a hospital setting, in a contagious environment, or in an undeveloped geographical area. Antibiotics or other therapies can be combined with the SeNPs. For example, antibiotics can target non-drug resistant bacteria while the SeNPs target drug resistant forms. The SeNPs also can be combined with radiation therapy or chemotherapy.

The mechanisms used by various microorganisms to reduce selenite are not completely elucidated and can vary. Some microorganisms are capable of reducing selenate (Se^{VI}O₄²⁻) and selenite (Se^{IV}O₃²⁻) oxyanions to Se⁰ as an elementary nanostructural form. These include bacteria isolated from areas contaminated with various pollutants including selenium compounds. The mechanisms used by bacteria to reduce selenites and selenates are diverse and may include one or several metabolic pathways and enzymes as well as other proteins. S. maltophilia, Bacillus sp, or Thauera sp can use selenites and selenates in their respiratory chain as electron acceptors, often along with sulfites and sulfates. It has been shown for several microorganisms that nitrate and nitrite reductases, which are responsible for denitrification, are involved in the reduction of Se^{IV} compounds. Consequently, selenite reduction may occur under the action of either nitrate reductase or nitrite reductase. Thus, the pathways to reduce selenites, selenates, and tellurites are often linked to denitrification.

Some bacteria can reduce Se^{IV} to selenium nanoparticles (SeNPs) under either aerobic or anaerobic conditions. Research has suggested that Se^{IV} reduction is involved in three different pathways: (1) the periplasmic nitrite reductase; (2) redox precipitation of both elemental sulfur and elemental Se; (3) a glutathione (GSH) reductase catalyzed reaction of GSH with Se (IV) to produce GS-Se-SG, further to generate GS-Se. Periplasmic nonspecific selenite reductases were involved in the reduction of selenite to SeNPs. These reductases mainly include nitrite reductase, sulfite reductase, and GSH reductase.

Once generated, these SeNPs can be used as antibacterial agents with low cytotoxicity towards healthy human cells. Examples tested include the environmentally safe synthesis of SeNPs using Escherichia coli, multidrug-resistant Escherichia coli, Pseudomonas aeruginosa, methicillin-resistant Staphylococcus aureus, and Staphylococcus aureus. The SeNPs are characterized and tested for their ability to inhibit bacterial growth. Inhibition, based on measurement of growth after 24 hours can be shown against both Gram-positive and negative bacteria at concentrations up to about 250 μg/mL. Similarly, SeNPs synthesized by Gram-negative Stenotrophomonas maltophilia, and Gram-positive Bacillus mycoides are active at low minimum inhibitory concentrations against a number of clinical isolates of Pseudomonas aeruginosa. As used herein, minimum inhibitory concentration (MIC) is the lowest concentration of an antibacterial agent that inhibits the visible growth of a bacterium after overnight incubation. Dendritic cells and fibroblasts exposed to the SeNPs do not show loss of cell viability, increase in the release of reactive oxygen species (ROS), or significant increase in the secretion of pro-inflammatory and immunostimulatory cytokines.

The selectivity of bacteriogenic nanoparticles poses fundamental questions, and the present technology can provide distinct selectivity. Metal-based nanoparticles typically are not species-specific, and this has resulted in concerns over concentration-dependent gained resistance. The present technology yields selenium-based nanostructures with the ability to kill bacteria selectively. Selective behavior can be obtained without extensive functionalization, subsequent characterization, and the employment of expensive reagents and capping agents.

The SeNPs disclosed herein can selectively inhibit the growth of the bacteria in which they were formed relative to the growth of other types of bacteria, even closely related bacteria. The bacteria used to synthesize the SeNPs, or “first bacteria” in the method, can be a drug resistant form of bacteria, whose non-drug-resistant form, or “second bacteria” are less potently inhibited or not inhibited by the SeNPs. By “type” of bacteria is meant a species, strain, or other population of bacteria having similar genetic and biochemical properties, such as metabolism, pathogenicity, or drug resistance. The present technology can provide selenium nanoparticles produced by a process including growing a first type of bacteria in the presence of a selenium salt which leads to selenium ions in the selenium salt being reduced to elemental selenium nanoparticles (SeNPs).

The present technology can provide methods of treating a subject for infection caused by drug-resistant bacteria, for cancer, or for combinations of ailments. The methods can include treating the subject by administering pharmaceutical composition with SeNPs, either alone or in combination with other therapies. The methods can include isolation of a drug-resistant bacteria from a subject and utilizing the drug-resistant bacteria to produce SeNPs for the subject or a different subject.

The biogenic SeNPs described herein can be used as therapies alone or in association with traditional antibiotics, to inhibit the growth of pathogenic bacteria. The SeNPs can provide drug delivery, imaging, and biosensors. For example, the SeNPs can be utilized to deliver a small molecule drug to a specifically targeted cell type. The SeNPs disclosed herein and the methods herein can include various radioisotopes during synthesis or applied after synthesis. Some non-limiting examples of radioisotopes used in some medical applications are strontium-92, selenium-75, molybdenum-99, technetium-99, bismuth-213, chromium-51, cobalt-60, copper-64, dysprosium-165, erbium-169, holmium-166, iodine-125, iridium-192, iron-59 (46 d), lutetium-177, palladium-103, phosphorus-32, potassium-42, rhenium-186, rhenium-188, samarium-153, sodium-24, strontium-89, xenon-133, ytterbium-169, yttrium-90, radioisotopes produced in cyclotrons, carbon-11, nitrogen-13, oxygen-15, fluorine-18, radioisotopes of caesium, gold, and ruthenium, cobalt-57, gallium-67, indium-111, iodine-123, krypton-81, and rubidium-82.

While the applications of the SeNPs presented herein, along with the methods, provide advances in medicine, the SeNPs can be utilized for many technologies. An example would be the use of SeNPs as catalysts. As used herein, the term “catalyst” refers to a component that directs, provokes, or speeds up a chemical reaction, for example, the reactions of a cell or of an industrial scale reaction. As used herein, a nanocarrier is a nanoparticle that can provide an agent to a cell; preferably a nanocarrier is specific to targeting a specific cell. The SeNPs herein can provide nanocarriers.

The technology for SeNPs herein can provide uniform size nanoparticles. The technology for SeNPs and methods provided herein can provide a selenium nanoparticle with layers or with shells. For example, one metal can be utilized to form a core of a nanoparticle and reaction conditions can be changed, during synthesis, to provide another layer (or composition) building upon the core of the nanoparticle. For example, another trace of a metal salt can be introduced mid-synthesis to change the composition of a hard-shell alloy, deposited upon the core of the nanoparticle, so long as the added ingredient does not inhibit growth of the synthesizing bacteria. Brief introduction of a radioisotope into the cultivation media can introduce a shell or layer with a radioisotope in the nanoparticle. So long as the synthesizing bacteria are growing and forming SeNPs, changes in cultivation conditions, nutrients, or bacteria can provide additional layers in the nanoparticles herein. The technology can provide a partial or complete outer coating on the nanoparticles, which can be referred to as a soft shell. Additives can be incorporated into the soft shell either during synthesis or after. The soft shell material can be made of organic material, and in particular, an organic material that includes at least one material capable of forming a coating specific for another bacteria or cancer cell. Non-limiting examples of organic soft shell materials include, but are not limited to, proteins, glycoproteins, antibodies, organic surfactants, organic or organic molecule-containing polymers, non-surfactant organic molecules, and combinations thereof.

As used herein, the term “nanostructure” refers to a structure having at least one dimension on the nanoscale, that is, at least one dimension between about 1 and 1000 nm, or in some cases between about 0.1 nm and 100 nm. Nanostructures can include, but are not limited to, nanoparticles, nanospheres, and combinations thereof. Aggregated nanostructures may be made up of a plurality of differently shaped or similarly shaped nanostructures. A nanostructure may have one dimension, such as thickness, on the nanoscale, with other dimensions, such as length, on the micro or millimeter scale. The term “microparticle” or “microstructure” refers to any particle having at least one dimension on the micrometer scale.

As used herein, the term “about” includes values close to the stated value as understood by one of ordinary skill. For example, the term “about” can refer to values within 10%, 5%, or 1%, of the stated value.

### BRIEF DESCRIPTION OF THE DRAWINGS

FIG. 1A shows UV-visible analysis (200-800 nm, N=3, 20 nm spacing) of selenium nanoparticles (SeNPs) produced after initial concentration of 2 mM sodium selenite cultured with E. coli. Media and bacterial culture absorbance values were subtracted to only show the contribution of selenium to the reaction development. FIG. 1B shows UV-visible analysis (200-800 nm, N=3, 20 nm spacing) of selenium nanoparticles (SeNPs) produced after initial concentration of 2 mM sodium selenite cultured with S. aureus. Media and bacterial culture absorbance values were subtracted to only show the contribution of selenium to the reaction development.

FIG. 2A shows kinetic analysis (48 hours, N=3) for E. coli in the presence of concentrations of sodium selenite at 0 mM (control), 1 mM, 2 mM, 3 mM, 5 mM, and 10 mM. FIG. 2B shows kinetics analysis (48 hours, N=3) for S. aureus in the presence of concentrations of sodium selenite at 0 mM (control), 1 mM, 2 mM, 3 mM, 5 mM, and 10 mM.

FIG. 3A shows a transmission electron microscopy (TEM) image of selenium nanoparticles (SeNPs) synthesized by multidrug-resistant (MDR) E. coli before purification. FIG. 3B shows a TEM image of SeNPs synthesized by methicillin-resistant Staphylococcus aureus (MRSA) before purification. FIG. 3C shows a TEM image of SeNPs synthesized by E. coli after purification. FIG. 3D shows a TEM image of SeNPs synthesized by S. aureus after purification.

FIG. 4A shows TEM characterization of SeNPs synthesized by E. coli with 2 mM of metallic salt concentration. FIG. 4B shows TEM characterization of SeNPs synthesized by S. aureus with 2 mM of metallic salt concentration.

FIG. 5A shows energy-dispersive X-ray spectroscopy (EDX) characterization of SeNPs synthesized by E. coli FIG. 5B shows EDX characterization of SeNPs synthesized by MDR E. coli.

FIG. 6A shows EDX characterization of SeNPs synthesized by S. aureus (SA). FIG. 6B shows EDX characterization of SeNPs synthesized by MRSA.

FIG. 7A shows a scanning electron microscope (SEM) image of MDR E. coli producing SeNPs. FIG. 7B shows an SEM image of MDR E. coli (EC) producing SeNPs. FIG. 7C shows an SEM image of MRSA producing SeNPs. FIG. 7D shows an SEM image of MRSA producing SeNPs.

FIG. 8 shows X-ray diffraction (XRD) patterns for EC-SeNPs (a, top trace), SA-SeNPs (b), MDR-EC-SeNPs (c), and MRSA-SeNPs (d, bottom trace).

FIG. 9 shows FT-IR spectra for EC-SeNPs (a, top), MDR-EC-SeNPs (b), SA-SeNPs (c), and MRSA-SeNPs (d, bottom). The FT-IR spectra were acquired in attenuated total reflectance (ATR) mode. The samples for (ATR) FT-IR analysis were prepared by drop casting the Se nanostructure colloids on a sample holder heated at ˜50° C. The IR spectra were scanned in the range of 500 to 4000 cm⁻¹ with a resolution of 4 cm⁻¹.

FIG. 10A shows TEM characterization of SeNPs synthesized by S. aureus with 2 mM of metallic salt concentration after 60 days. FIG. 10B shows TEM characterization of SeNPs synthesized by E. coli with 2 mM of metallic salt concentration after 60 days.

FIG. 11A shows a colony counting assay of E. coli (EC) after being treated for 8 hours with EC bacteria-mediated synthesized nanoparticles (EC-SeNPs). Data=mean+/−SEM, N=3, *p<0.05 versus control (0 μg/mL concentration), **p<0.01 versus control (0 μg/mL concentration). FIG. 11B shows a colony counting assay of E. coli (EC) after being treated for 8 hours with MDR-E. coli bacteria-mediated synthesized nanoparticles (MDR-SeNPs). Data=mean+/−SEM, N=3, *p<0.05 versus control (0 μg/mL concentration), **p<0.01 versus control (0 μg/mL concentration). FIG. 11C shows a colony counting assay of MDR-E. coli after being treated for 8 hours with MDR-E. coli bacteria-mediated synthesized nanoparticles (MDR-SeNPs). Data=mean+/−SEM, N=3, *p<0.05 versus control (0 μg/mL concentration), **p<0.01 versus control (0 μg/mL concentration). FIG. 11D shows a colony counting assay of MDR-E. coli after being treated for 8 hours with EC bacteria-mediated synthesized nanoparticles (EC-SeNPs). Data=mean+/−SEM, N=3, *p<0.05 versus control (0 μg/mL concentration), **p<0.01 versus control (0 μg/mL concentration).

FIG. 12A shows a colony counting assay of S. aureus (SA) after being treated for 8 hours with SA bacteria-mediated synthesized nanoparticles (SA-SeNPs). Data=mean+/−SEM, N=3, *p<0.05 versus control (0 μg/mL concentration), **p<0.01 versus control (0 μg/mL concentration). FIG. 12B shows a colony counting assay of S. aureus (SA) after being treated for 8 hours with methicillin-resistant S. aureus (MRSA) bacteria-mediated synthesized nanoparticles (MRSA-SeNPs). Data=mean+/−SEM, N=3, *p<0.05 versus control (0 μg/mL concentration), **p<0.01 versus control (0 μg/mL concentration). FIG. 12C shows a colony counting assay of MRSA after being treated for 8 hours with MRSA-SeNPs. Data=mean+/−SEM, N=3, *p<0.05 versus control (0 μg/mL concentration), **p<0.01 versus control (0 μg/mL concentration). FIG. 12D shows a colony counting assay of MRSA after being treated for 8 hours with S. aureus (SA) bacteria-mediated synthesized nanoparticles (SA-SeNPs). Data=mean+/−SEM, N=3, *p<0.05 versus control (0 μg/mL concentration), **p<0.01 versus control (0 μg/mL concentration).

FIG. 13A shows a colony counting assay of MDR-EC after being treated for 8 hours with MRSA-SeNPs. Data=mean+/−SEM, N=3, *p<0.05 versus control (0 μg/mL concentration), **p<0.01 versus control (0 μg/mL concentration). FIG. 13B shows a colony counting assay of MDR-EC after being treated for 8 hours with SA-SeNPs. Data=mean+/−SEM, N=3, *p<0.05 versus control (0 μg/mL concentration), **p<0.01 versus control (0 μg/mL concentration). FIG. 13C shows a colony counting assay of MRSA after being treated for 8 hours with MDR-SeNPs (MDR-EC-SeNPs). Data=mean+/−SEM, N=3, *p<0.05 versus control (0 μg/mL concentration), **p<0.01 versus control (0 μg/mL concentration). FIG. 13D shows a colony counting assay of MRSA after being treated for 8 hours with EC bacteria-mediated synthesized nanoparticles (EC-SeNPs). Data=mean+/−SEM, N=3, *p<0.05 versus control (0 μg/mL concentration), **p<0.01 versus control (0 μg/mL concentration).

FIG. 14A shows a colony counting assay of EC after being treated for 8 hours with MRSA-SeNPs. Data=mean+/−SEM, N=3, *p<0.05 versus control (0 μg/mL concentration), **p<0.01 versus control (0 μg/mL concentration). FIG. 14B shows a colony counting assay of EC after being treated for 8 hours with SA-SeNPs. Data=mean+/−SEM, N=3, *p<0.05 versus control (0 μg/mL concentration), **p<0.01 versus control (0 μg/mL concentration). FIG. 14C shows a colony counting assay of S. aureus (SA) after being treated for 8 hours with MDR-SeNPs (MDREC-SeNPs). Data=mean+/−SEM, N=3, *p<0.05 versus control (0 μg/mL concentration), **p<0.01 versus control (0 μg/mL concentration). FIG. 14D shows a colony counting assay of SA after being treated for 8 hours with EC bacteria-mediated synthesized nanoparticles (EC-SeNPs). Data=mean+/−SEM, N=3, *p<0.05 versus control (0 μg/mL concentration), **p<0.01 versus control (0 μg/mL concentration).

FIGS. 15A-15D show MTS assay on human dermal fibroblast (HDF) in the presence of MRSA-SeNPs (FIG. 15A), MDR-SeNPs (FIG. 15B), SA-SeNPs (FIG. 15C) and EC-SeNPs (FIG. 15D) ranging from 25 to 100 μg/mL. Data=mean+/−SEM, N=3, *p<0.05 versus control (0 μg/mL concentration), **p<0.01 versus control (0 μg/mL concentration).

FIGS. 16A-16D show MTS assay on human melanoma cells in the presence of MRSA-SeNPs (FIG. 16A), MDR-SeNPs (FIG. 16B), SA-SeNPs (FIG. 16C) and EC-SeNPs (FIG. 16D) ranging from 25 to 100 μg/mL. Data=mean+/−SEM, N=3, *p<0.05 versus control (0 μg/mL concentration), **p<0.01 versus control (0 μg/mL concentration).

FIG. 17A shows an SEM image of control MDR E. coli without treatment with SeNPs. FIG. 17B shows an SEM image of MDR E. coli after treatment with MDR-SeNPs (MDR-EC SeNPs). FIG. 17C shows an SEM image of control MRSA without treatment with SeNPs. FIG. 17D shows an SEM image of MRSA after treatment with MRSA-SeNPs.

FIGS. 18A-18D show ROS (reactive oxygen species) study of MRSA-SeNPs analysis (FIG. 18A), MDR-SeNPs analysis (FIG. 18B), SA-SeNPs analysis (FIG. 18C) and EC-SeNPs analysis (FIG. 18D).

### DETAILED DESCRIPTION

The present technology provides methods for selectively inhibiting the growth of a bacterial pathogen by contacting the pathogen with nanoparticles containing elemental selenium, referred to herein as selenium nanoparticles or SeNPs. The SeNPs can be produced by a process including growing a first type of bacteria in the presence of a selenium salt wherein selenium ions in the selenium salt are reduced to elemental selenium. The first type of bacteria in the production process can be the same as the targeted bacterial pathogen, which can be a drug resistant form. The production process can provide a partial or full coating with the SeNPs that provides for selectivity and other properties, for example, stability. The SeNPs provide methods for inhibiting cancer cell growth by contacting a cancer cell with SeNPs. As distinct from nanoparticles synthesized chemically, SeNPs of the present technology are microbiologically produced and can contain proteins and other bioorganic constituents. The proteins are associated with elemental selenium and are believed to have a role in stabilizing the SeNPs as well as a role in the unique selectivity. Also provided are pharmaceutical compositions with the SeNPs and a method for treating bacterial infection in a subject using the pharmaceutical composition.

The elemental selenium nanoparticles are produced by a process including growing a first type of bacteria in the presence of a selenium salt. This results in the selenium ions present in the selenium salt being reduced to elemental selenium in the form of SeNPs. The elemental Se in the SeNPs can be in various crystal forms. Salts of selenous acid (H₂SeO₃) are called selenites. Examples include silver selenite (Ag₂SeO₃) and sodium selenite (Na₂SeO₃). The mechanisms used by various bacteria to reduce selenite are not completely understood. Several metabolic pathways, enzymes, and proteins may be involved. For several bacterial strains, the reduction of selenite may occur under the action of nitrate enzymes, as with E. coli, or nitrite reductases. Therefore, the reduction of selenite is usually linked to denitrification in bacteria. Furthermore, thiols, such as glutathione (GSH), present inside the cytoplasm of bacterial cells, are involved in the reduction of selenite ions.

In Example 1 it is shown that E. coli and S. aureus, in their standard and antibiotic-resistant phenotypes, are able to transform toxic selenite ions in the media to insoluble and non-toxic SeNPs, through a process of detoxification. The proposed mechanism for the production of nanoparticles can be divided into four main processes: (1) transport of selenite anions inside the cells (intracellular synthesis), (2) redox reaction inside or outside the cells, (3) export of nanometric selenium out of the cells (if the ions penetrated inside the cell), and (4) assembly and aggregation of nanoparticles. Despite the complex system of enzymatic reactions involved in the production of nanospheres and nanoparticles (SeNPs), the process can be described in one elementary redox reaction:

Se^{IV}O₃²⁻+4e⁻+6H⁺→Se+3H₂O Equation 1. Example Overall Reaction for Selenite Reduced to Selenium.

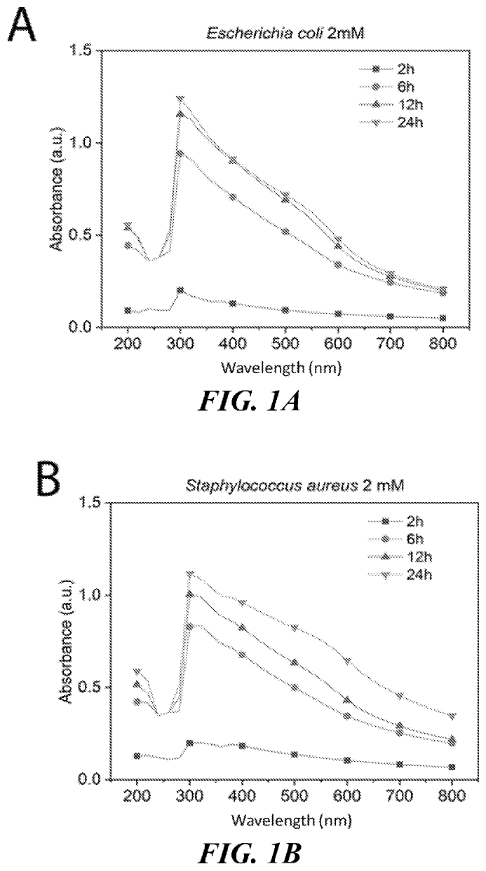

The production of SeNPs by bacteria can be within a short time. Example 2 demonstrates UV-visible monitoring of E. coli and S. aureus production of SeNPs. In the UV-vis. data shown in FIG. 1A and FIG. 1B, media and bacterial culture absorbance values are background subtracted to only show the contribution of selenium to the reaction development. The peak for selenium increases from 2 to 6 hours with a slight further increase for up to 24 hours. In the growth conditions of Example 2, which are initiated with 2 mM selenite, no further increase was observed when the UV-vis. measurements were continued up to 48 hours, so the 48 hour data is not shown in FIGS. 1A and 1B. The kinetics of the bacterial response to the presence of different starting concentrations of sodium selenite are further investigated in Example 3.

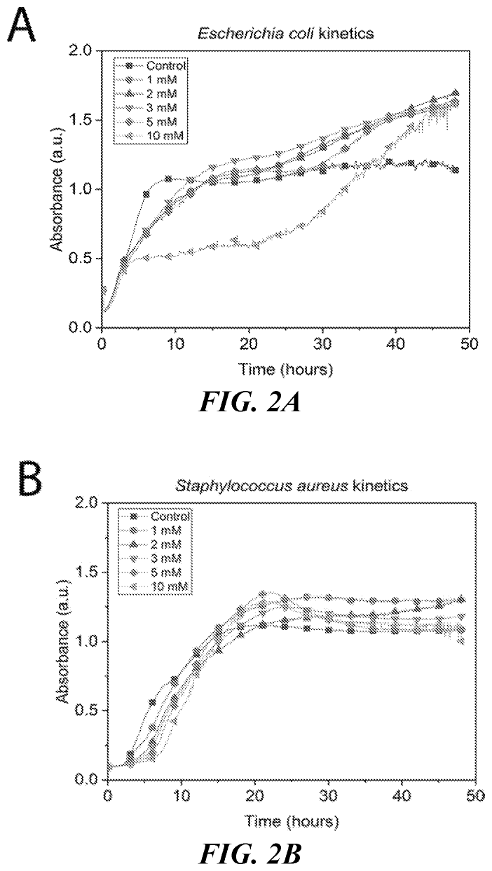

In Example 3, low starting concentrations of selenite show delayed bacterial growth, compared to normal bacterial (control) growth rate. FIG. 2A shows delayed growth of E. coli at about 10 hours for 1 mM, 2 mM, 3 mM, 5 mM, and 10 mM (starting) selenite concentrations, compared to the control, and FIG. 2B shows the same data for S. aureus. For bacteria growing with various concentrations of selenite, a toleration point can be reached. At the toleration point, the bacteria can start growing at the rate of the control bacteria. In FIG. 2A, 10 mM selenite concentration delays the bacterial growth, until a toleration point close to 25 hours. The delayed growth of bacteria growing in the presence of selenite is attributed to the toxicity of selenite ions in solution. The delay might be caused by the effort of bacterial cells to cope with the presence of these ions in solution. Bacteria spend time generating nanoparticles, a non-toxic form of selenium, instead of growing, explaining why the nutrient level shows a later decay for bacteria generating nanoparticles, prolonging the life cycle of the bacteria longer than the one found in control.

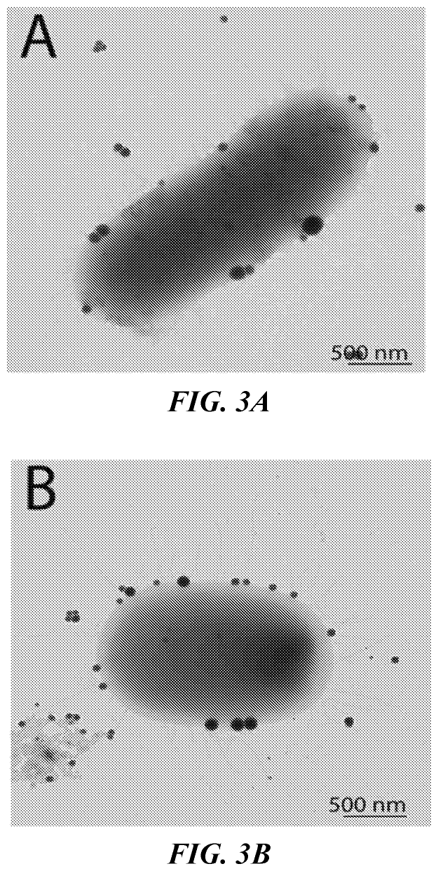

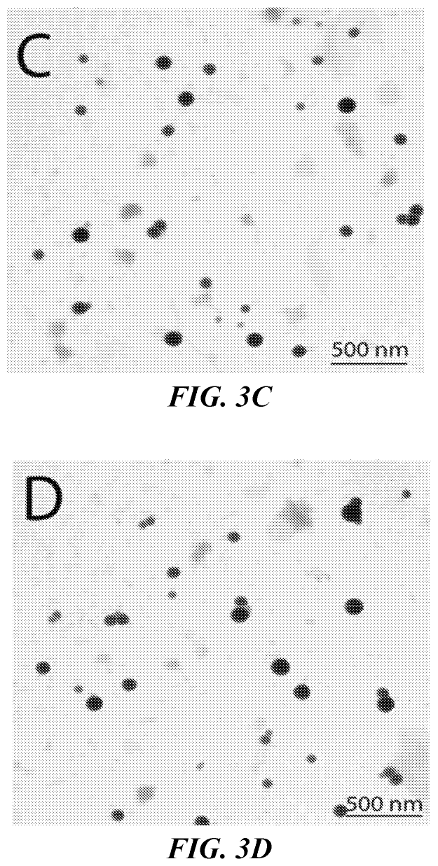

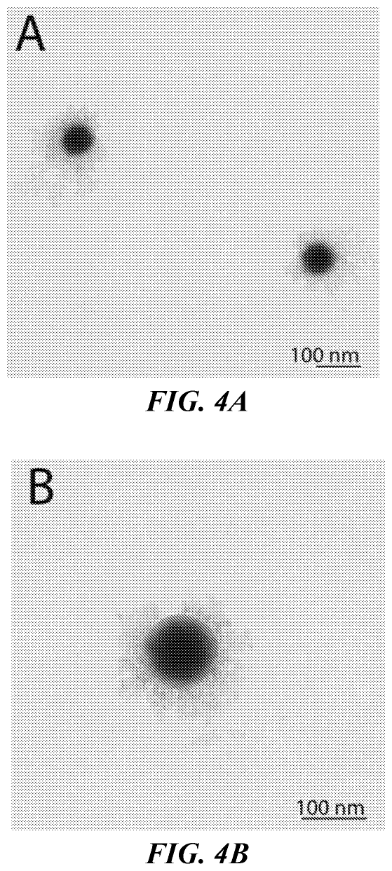

A thorough morphological study of the SeNPs is presented in Example 4 with TEM, SEM, EDX, XRD, and FTIR experiments. Synthesized SeNPs are seen in the TEM (transmission electron microscopy) images shown in FIG. 3A (with E. coli) and FIG. 3B (with MRSA, methicillin-resistant S. aureus). As seen in FIGS. 3A and 3B, the nanoparticles can be synthesized inside and outside the cells. Small nanospheres can be found within the cytoplasm, while bigger nanoparticles can be found in the outer surface of the external membrane. From the TEM images (Example 4), it is quite difficult to know if the nanoparticles that appear within the external membrane are synthesized inside or outside the cells. The aggregation of small clusters in the inner part of the membrane close to some of the nanospheres might indicate that there is a movement of the nuclei to the external part of the cells and they are assembled to nanospheres in those areas. The SeNPs are shown, in a TEM image after purification, in FIGS. 3C and 3D. Once the nanoparticles are released from the cells after purification, they are able to remain relatively monodispersed in solution due to the action of an organic coating (FIGS. 4A and 4B) that surrounds the nanospheres once they abandon the bacterial matrix.

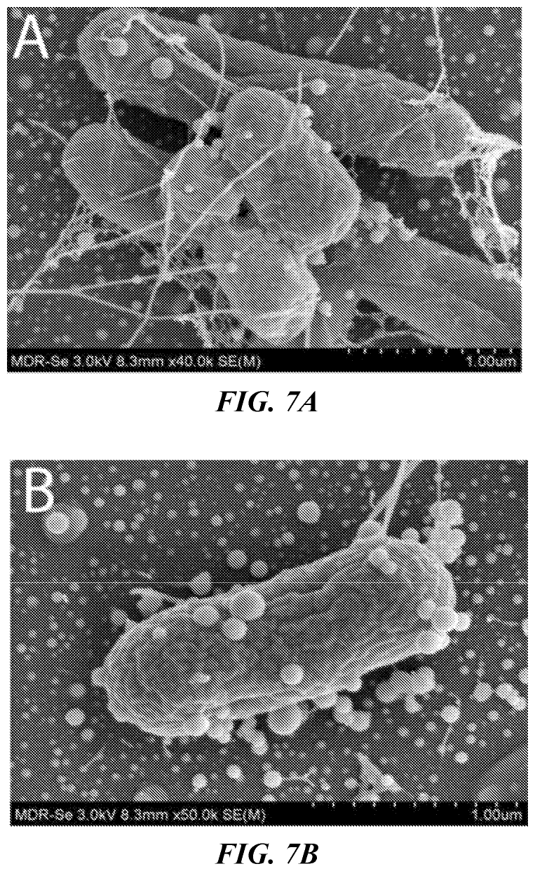

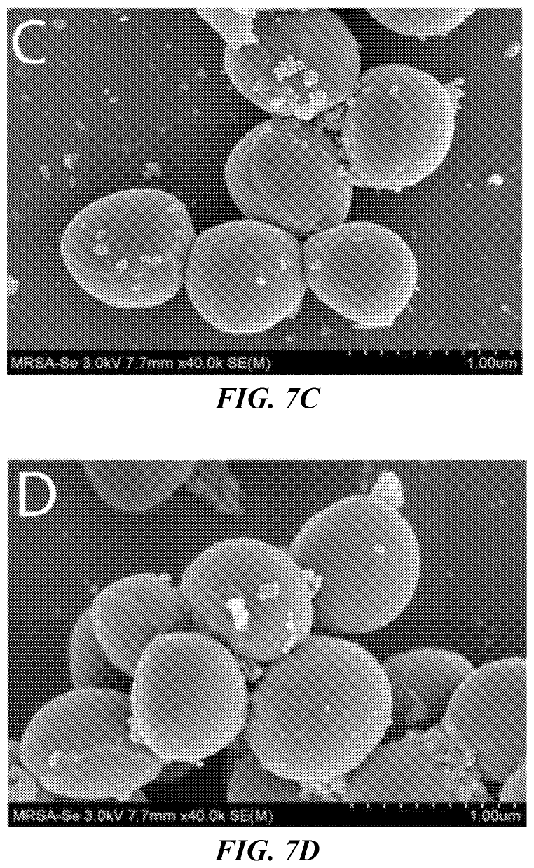

Cell fixation combined with SEM (scanning electron microscopy) shows the synthesis of the SeNPs once the synthetic protocol was completed (24 hours after inoculation of the Se salt) in FIGS. 7A and 7B (multidrug-resistant E. coli or MDR E. coli), also in FIGS. 7C and 7D (showing MRSA). After 24 hours, the MDR E. coli are able to generate relatively monodispersed and uniformly sized selenium nanospheres that are spread all over the surface of the cells, with no apparent disruption of the cellular membranes (see FIGS. 7A and 7B). In contrast, MRSA bacteria form smaller nanoparticles that are present all over the surface of the membranes, with some level of aggregation observed in the space between cells (see FIGS. 7C and 7D).

SeNPs of the present technology are microbiologically produced and can contain proteins and other bioorganic constituents. As mentioned above, proteins associated with SeNPs are thought to have a role in stabilizing the nanoparticles. This coating (or corona) can be made of biomolecules coming from the bacterial cells and can assemble to the outer layer of the SeNPs, as can be seen for nanoparticles made by both E. coli (FIG. 4A) and S. aureus (FIG. 4B).

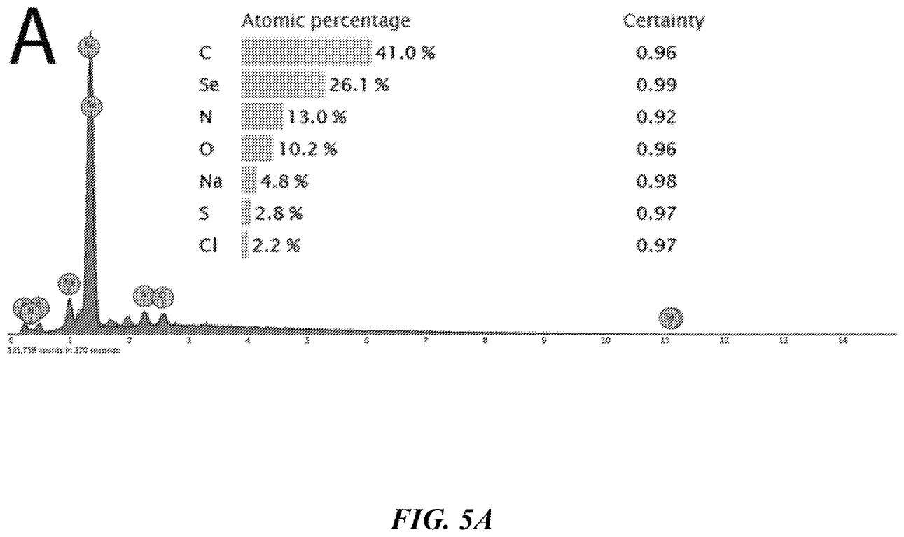

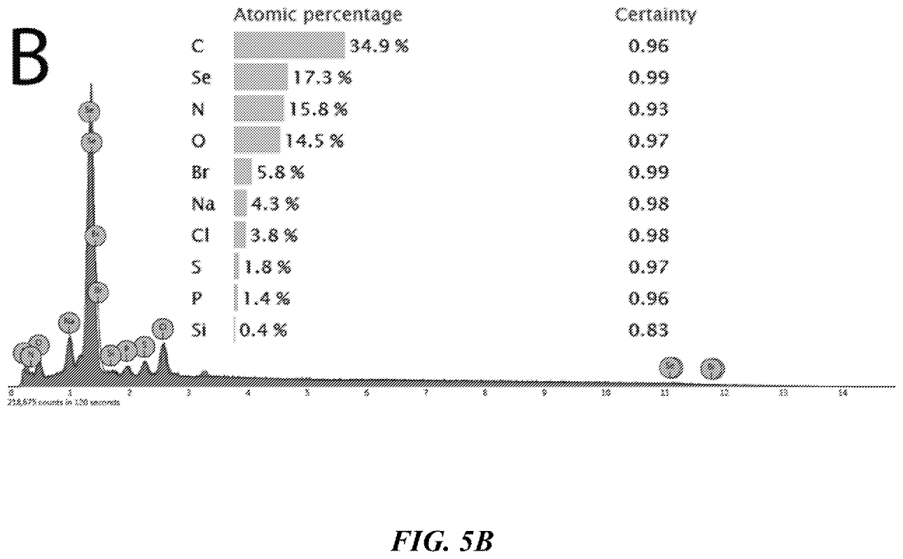

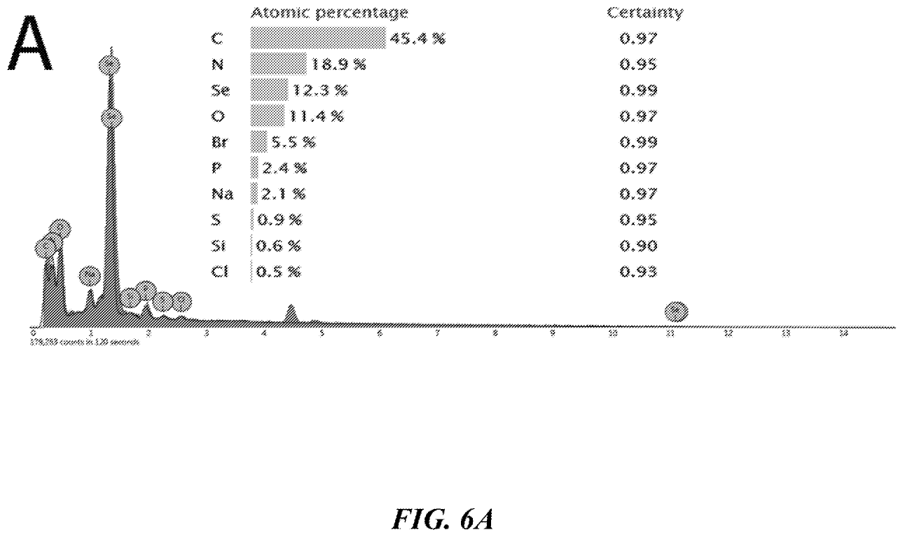

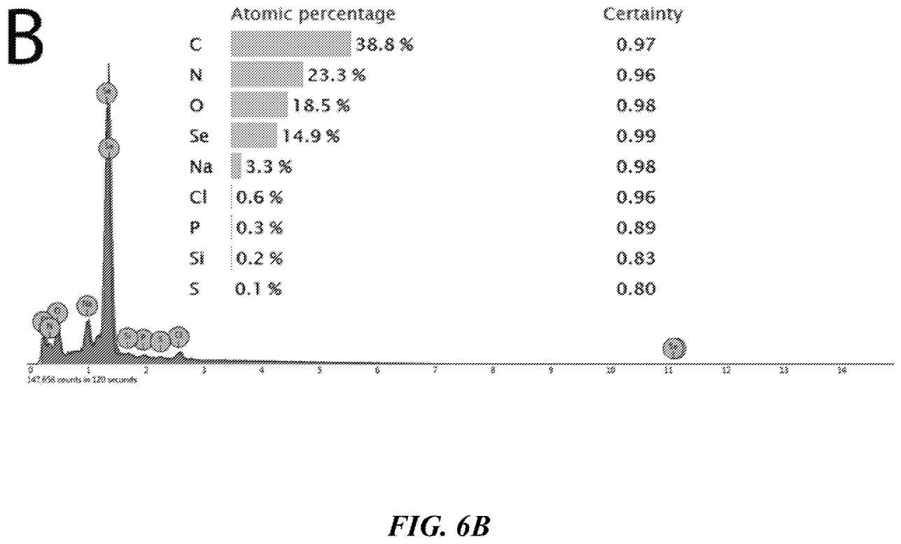

The EDX spectra in FIGS. 5A, 5B, 6A, and 6B show a high content of selenium, as well as carbon, oxygen, and nitrogen, indicating the presence of the organic coating surrounding the SeNPs. EDX characterization shows higher organic contribution in the spectra for SA-SeNPs (S. aureus-SeNPs, FIG. 6A) and MRSA-SeNPs (FIG. 6B) than in the spectra for EC-SeNPs (FIG. 5A) and MDR-EC-SeNPs (FIG. 5B). These results may be derived from the smaller size of the SeNPs synthesized by the Gram-positive (SA, S. aureus) bacteria that are able to form small aggregated particles embedded in organic matter (e.g., FIGS. 7C and 7D).

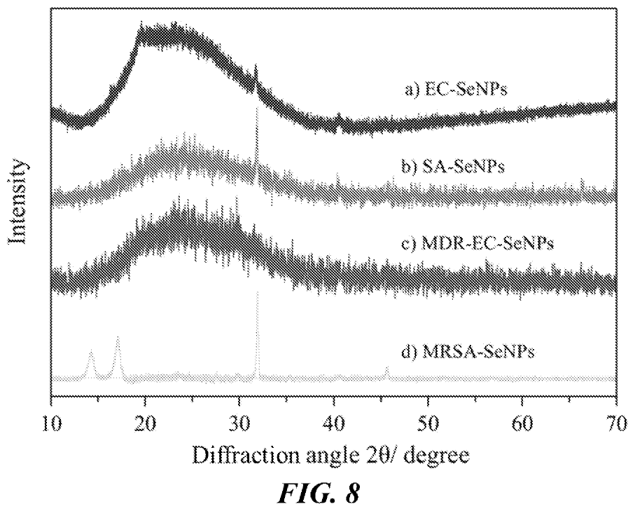

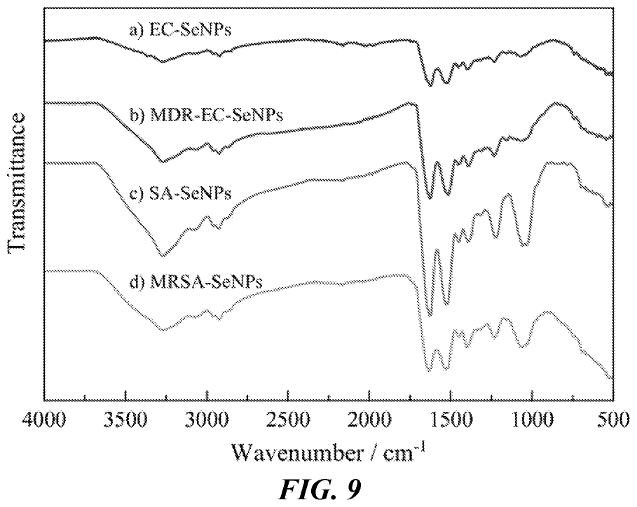

Crystallinity is studied in FIG. 8 by X-ray diffraction, (Example 4). The XRD peaks for the MRSA-SeNPs' XRD pattern (bottom spectrum, FIG. 8) may be indexed to trigonal Se structure (α-Se, space group P3121). The XRD spectra for EC-SeNPs, SA-SeNPs, and MDR-EC-SeNPs, (see FIG. 8) show a more abundant amorphous phase than in the sample for MRSA-SeNPs, but in the spectra for EC-SeNPs and SA-SeNPs, the most intense peak is located at diffraction of 2Θ=31°, which is closely related to trigonal selenium. The methods disclosed herein can provide selenium nanoparticles in various crystal forms, or with varying amounts of amorphous, depending on the microorganism used for the synthesis and the synthetic conditions. FTIR data (acquired in ATR mode) for exemplary SeNPs is presented in FIG. 9 and described in more detail in Example 4, while stability of the SeNPs is studied in Example 5. The stability demonstrates the SeNPs are suitable for formulation development, for example, a pharmaceutical formulation for administration. The Z-potential (mV), as synthesized can be higher in magnitude than about ±30 mV (see Example 5).

The present technology can provide SeNPs with properties determined by the type of bacteria used to produce the SeNPs and by the growth conditions. For example, the XRD data and the EDX data support differences in core and shell (coating) of the SeNPs, respectively. Based on the XRD data (FIG. 8), MDR-EC-SeNPs (or MDR-SeNPs) show an amorphous halo without readily discernable XRD peaks. Accordingly, production of amorphous SeNPs can be done with MDR-EC, and SeNPs with varying amounts of crystalline form can be synthesized. Production of crystalline SeNPs can be done with MRSA (FIG. 8, bottom trace). The methods provided herein enable custom-designed SeNPs, for example, by selecting type of bacteria, by introducing different growth conditions or additives, or by applying energy (e.g., microwaves) to the synthesis reaction after formation of nanoparticles.

Examples of size ranges for the synthesized SeNPs can be from about 10 nm to about 250 nm, from about 40 nm to about 80 nm, from about 50 nm to about 90 nm, from about 50 nm to about 75 nm, from about 70 nm to about 110 nm, from about 70 nm to about 130 nm, and about 80 nm to about 140 nm, depending on the microorganism and the conditions.

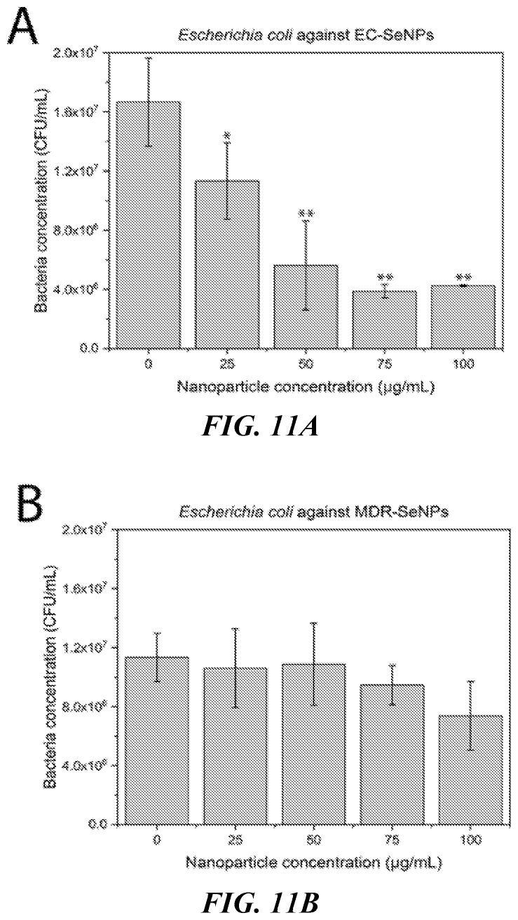

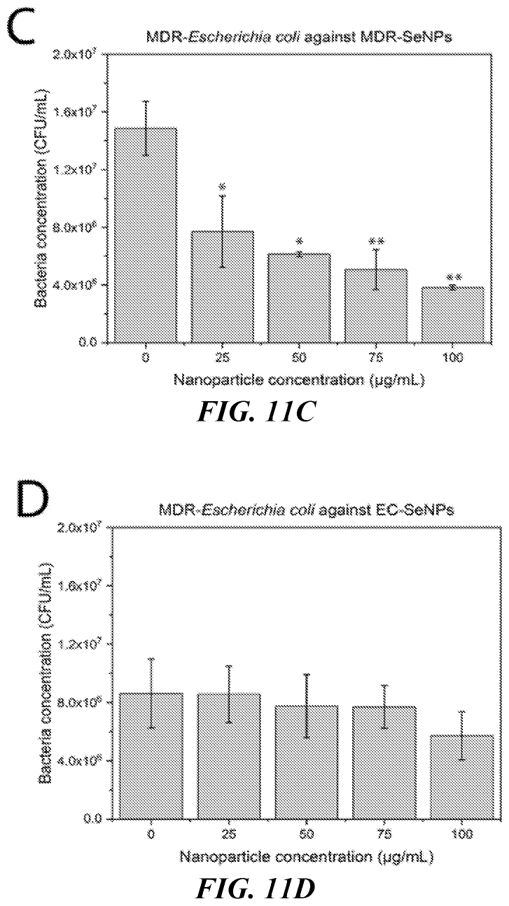

The properties of the SeNPs deliver antimicrobial effects and antimicrobial methods for drug resistant bacteria that are unforeseen. For example, in FIG. 11A, E. coli (EC) cultured with EC-SeNPs shows inhibition of growth, and in FIG. 11C, MDR-EC cultured with MDR-SeNPs (8 hours) shows inhibition of growth. In Example 6, a “straight analysis” is presented wherein SeNPs made by one type of bacteria are tested against the same type of bacteria, and a “crossed analysis” is presented wherein SeNPs made by one type of bacteria are tested against a different type. The straight analyses show significant inhibition of drug resistant bacteria. Surprisingly, the SeNPs can also inhibit growth of cancer cells.

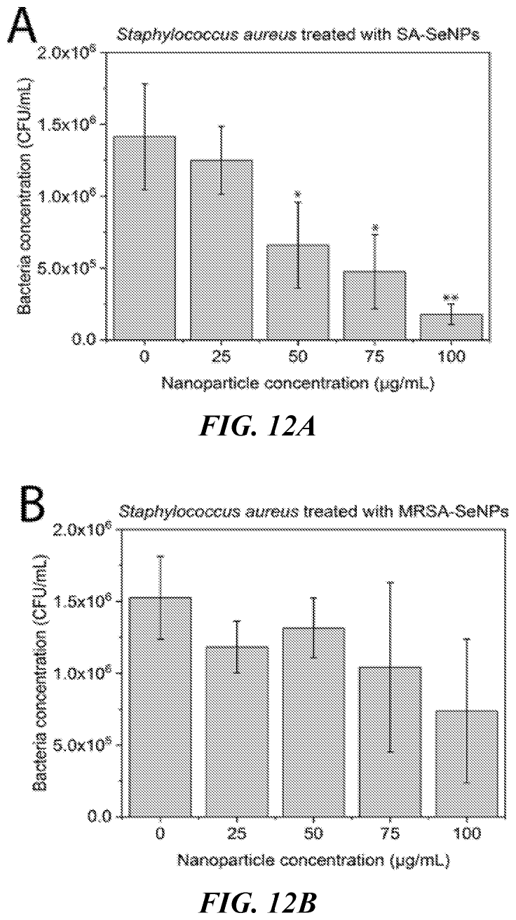

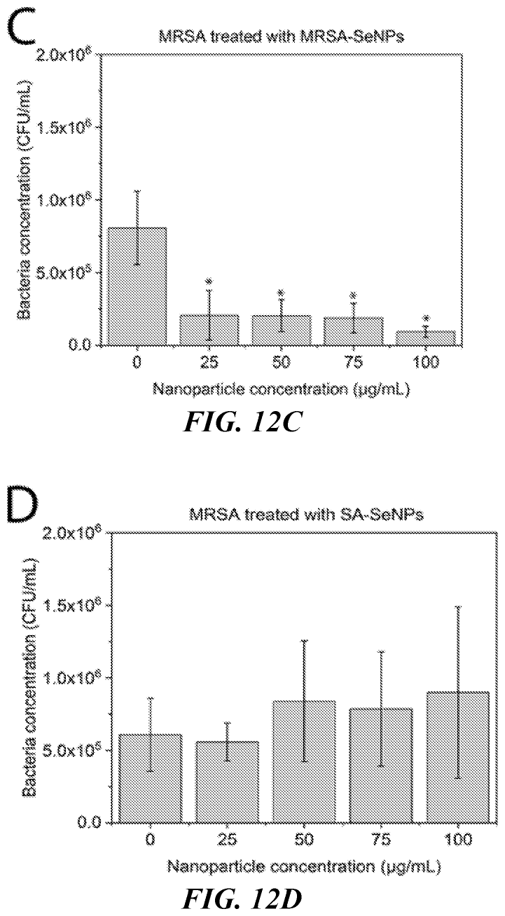

The straight analysis for E. coli (FIGS. 11A and 11B) and for MDR E. coli (FIGS. 11C and 11D) shows a dose-dependent inhibition of the bacteria when they were cultured with EC-SeNPs and with MDR-SeNPs. For MDR-SeNPs, the concentrations between 25 to 100 μg/mL show the antibacterial effects for MDR E. coli bacteria (FIG. 11C). The straight analysis for MRSA-SeNPs demonstrate dose-dependent inhibition of MRSA in FIG. 12C. Accordingly, a method of inhibiting growth of a drug resistant bacteria can include contacting the drug resistant bacteria with SeNPs produced by cultivating the drug resistant bacteria with a selenium salt or ion. The concentration of the SeNPs can be very low because the specificity of the SeNPs is high. The specificity can be due to the coating on the SeNPs, which is produced by the bacteria that produced the SeNPs.

For example, the MIC (μg/mL) of the SeNPs against various bacteria can be about 10 μg/mL, about 15 μg/mL, about 20 μg/mL, about 26 μg/mL, about 29 μg/mL, about 30 μg/mL, about 31 μg/mL, about 34 μg/mL, about 40 μg/mL, or about 45 μg/mL.

Surprisingly, growth of cancer cells can be inhibited by applying the present technology. Results from Example 7 demonstrate that bacteria-derived SeNPs can display selective antibacterial effects and good anticancer effects, with a negligible cytotoxic effect for healthy human cells within a range that can be about 25 μg/mL. Without being limited by any theory or mechanism of action, it is believed that the biocompatibility of the metallic nanostructures is associated with the organic coating present over the SeNPs. This coating, composed of organic material coming from the bacteria, surrounding the metallic surface can prevent the ions release and avoid cell damage. The Examples support an enhancement of biocompatibility of green-synthesized nanoparticles towards chemically-synthesized nanoparticles, showing no significant cytotoxic effect on normal cells.

The SeNPs disclosed herein, for example when directed towards cancer cells, can have an IC₅₀ (24 hours) of about 5 μg/mL, about 7.5 μg/mL, about 8 μg/mL, about 10 μg/mL, about 12 μg/mL, about 15 μg/mL, about 20 μg/mL, or about 25 μg/mL. The IC₅₀ (72 hours) can be about 5 μg/mL, about 7.5 μg/mL, about 9 μg/mL, about 10 μg/mL, about 11 μg/mL, about 14 μg/mL, about 15 μg/mL, or about 20 μg/mL. A deteriorate ROS protective mechanism found in cancer cells may explain the observed anticancer effect. Example 9 presents data for ROS utilizing human melanoma cells.

Based on the inhibition of growth demonstrated for drug resistant bacteria and for cancer cells, the SeNPs disclosed herein can provide a method for inhibiting the growth of an abnormal cell, a resistant cell, a cancer cell, or a normal cell.

A method for synthesizing SeNPs can include growing a first type of bacteria in the presence of a selenium salt wherein selenium ions from the selenium salt are reduced to elemental selenium in the form of SeNPs. The SeNPs can have a partial or complete coating from the first type of bacteria. The SeNPs can selectively inhibit the growth of the first bacteria relative to the growth of a second type of bacteria, and wherein the first type of bacteria is a drug resistant form of the second type of bacteria. The method can be tuned or modified to produce different SeNPs, for example by adding other metal ions or additives that can be incorporated into the coating. Other modifications are seen by the present technology, for example the incorporation of radioisotopes for imaging, the incorporation of magnetic properties to enable magnetic sorting of the SeNPs or thermal-magnetic (therapeutic) properties in the SeNPs, or the use of genetically modified bacteria to synthesize the SeNPs. Another non-limiting example is the use of microwaves to purify, heat, or modify the SeNPs during or after synthesis. The methods provided by the technology can be modified to be continuous methods. For example, by implementing continuous separation of the SeNPs from the microorganisms and continuous introduction of new microorganisms.

The technology can provide a kit for inhibiting the growth of a drug resistant bacteria. The kit can have a selenium salt and instructions for cultivating a drug resistant bacteria with the selenium salt such that selenium ions from the selenium salt are reduced to elemental selenium.

The technology can provide a method of inhibiting the growth of a bacterial pathogen, the method including contacting the pathogen with elemental SeNPs. The elemental SeNPs can be produced by a process of growing a first type of bacteria in the presence of a selenium salt wherein selenium ions in the selenium salt are reduced to elemental selenium. The SeNPs can selectively inhibit the growth of the first bacteria relative to the growth of a second type of bacteria. The first type of bacteria can be a drug resistant form of the second type of bacteria. The first type of bacteria can be the bacterial pathogen. The SeNPs can be used to selectively deliver a payload to the bacterial pathogen, for example, an antibiotic or oligonucleotide. The selectivity can be because the elemental SeNPs are at least partially coated with an organic coating, the organic coating produced by the process of growing a first type of bacteria in the presence of a selenium salt.

For example, the second type of bacteria can be E. coli and the first type of bacteria can be a drug resistant form of E. coli. The second type of bacteria can be S. aureus and the first type of bacteria can be a drug resistant from of S. aureus. The minimum inhibitory concentration of the elemental selenium nanoparticles to a bacterial pathogen can be less than about 50 μg/mL, less than about 30 μg/mL, less than about 25 μg/mL, less than about 20 μg/mL, less than about 15 μg/mL, less than about 10 μg/mL, less than about 5 μg/mL, or less than about 3 μg/mL.

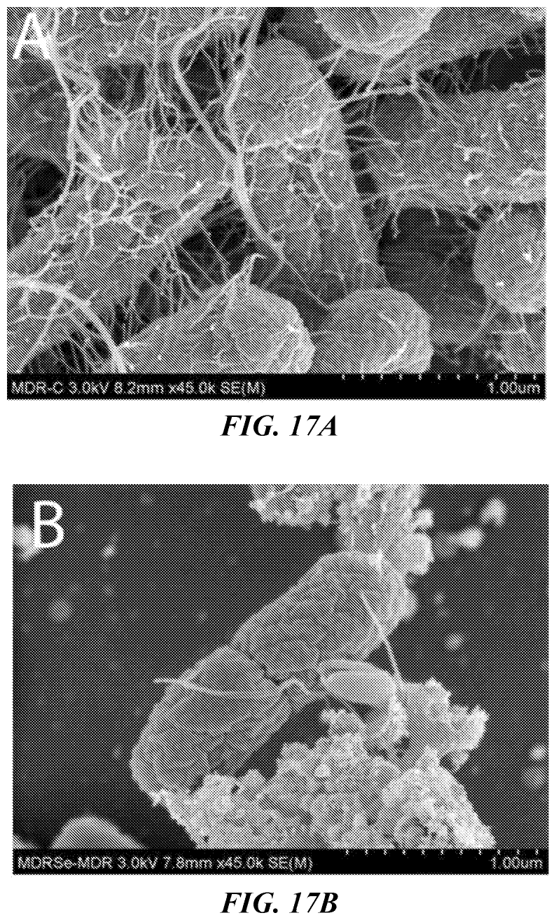

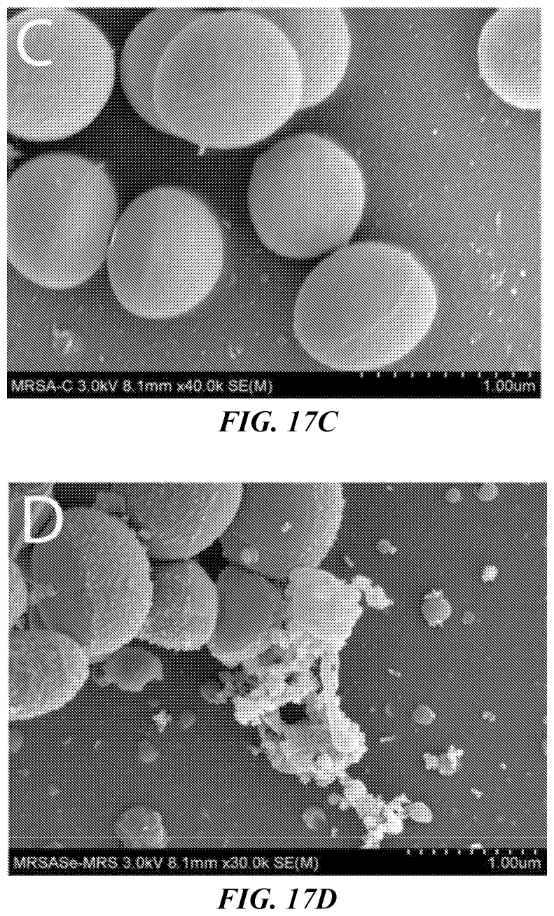

Example 8 shows disruption of outer cell membranes and cell lysis after treatment with SeNPs. The disruption of cell membranes shown (see FIGS. 17B and 17D) is commonly found to be a cause of ROS. Other mechanisms can be inferred, for example, the direct damage of the cells due to the morphology of the nanostructures. From the SEM images of the bacteria in FIGS. 17A-17D, it is possible to see that the membrane damage occurs and that there was the attachment of nanoparticles to bacteria, but the exact mechanism how damage occurs can vary. FIGS. 17A-17D show SEM micrographs of control MDR E. coli and MRSA (A, C) and bacteria after treatment with MDR-SeNPs and MRSA-SeNPs (B, D), respectively.

The technology can provide a kit for inhibiting the growth of cancerous cells. The kit can provide a selenium salt and instructions for cultivating a cell, microorganism, or bacteria in the presence of the selenium salt such as to produce Se nanoparticles.

The technology can provide a method of inhibiting the growth of a drug resistant bacteria in a living subject. The method can include administering SeNPs to the living subject in the form of a pharmaceutically acceptable formulation. The SeNPs can be produced by a process including growing the drug resistant bacteria, outside of the living subject, in the presence of a selenium salt wherein selenium ions in the selenium salt are reduced to elemental selenium. The method can be for inhibiting the growth of cancerous cells in a living subject, for example, administering SeNPs to the living subject in the form of a pharmaceutically acceptable formulation. The SeNPs for cancer inhibition can be synthesized by growing a microorganism in the presence of a selenium salt. The method can be wherein growth of non-cancerous cells in the living subject is not significantly inhibited.

The description and the following examples arise from data showing urgently needed effectiveness for drug resistant bacteria with a quickly adaptable method. The technology has other applications, as such the scope of the claimed technology is not limited by the applications disclosed herein.

### EXAMPLES

The experiments described below were carried out in triplicate (N=3) unless otherwise stated. Statistical significance was assessed using Student's t-test, with a p<0.05 being statistically significant. Results are displayed as mean±standard deviation.

### Example 1: Synthesis of Selenium Nanoparticles Utilizing Bacteria

Escherichia coli (strain K-12 HB101, Bio-Rad, Hercules, Calif.); Staphylococcus aureus (ATCC 12600TM); multidrug-resistant Escherichia coli (MDR E. coli) (ATCC BAA-2471, ATCC, Manassas, Va.); methicillin-resistant Staphylococcus aureus (MRSA) (ATCC 4330, ATCC, Manassas, Va.); and R aeruginosa (Schroeter, MIgula, ATCC, 27853) were used to synthesize selenium nanoparticles (SeNPs). Luria-Bertani Broth (LB) was purchased from Sigma-Aldrich (St Louis, Mo., US). The bacteria cultures were maintained on an agar plate at 48° C. For the inoculum preparation, a loop of the culture was inoculated into 40 mL sterile Luria-Bertani (LB) broth in a 50 mL conical centrifuge tube and incubated at 37° C. at 200 rpm for 24 hours. The bacteria were harvested by centrifugation at 6000 rpm for 10 min, upon which time the supernatant was collected and transferred into another 50 mL centrifuge tube. The pellet phase was collected and stored in a freezer for further experiments. The optical density of the supernatant phase optical density was measured using a spectrophotometer (SpectraMax M3, Molecular Devices, Sunnyvale, Calif.) at 600 nm (OD600), to estimate the number of bacterial cells per milliliter for further experiments.

For selenium nanoparticle synthesis, an aqueous solution of 2 mM sodium selenite (Na₂SeO₃) was added to 40 mL of supernatant. After inoculation, bacteria were kept in a shaking incubator at 37° C. and 200 rpm for 24 hours. For purification of nanoparticles, the samples were centrifuged at 7500 rpm for 10 minutes. The supernatant was removed, and 20 mL of DI-water was added into the tubes. The solutions were sonicated for 5 minutes to allow for disruption of the bacterial membranes followed by the release of the nanoparticles. After sonication, the samples were centrifuged at 10000 rpm for 30 minutes and re-suspended in 10 mL of sterile Milli-Q water. Then, these solutions were placed in a freezer at −80° C. for 4 hours and after that, kept in a freeze-dryer overnight. The powder obtained was weighed and re-suspended in Milli-Q water to a known concentration for further experiments.

An in-depth study of the synthesis of SeNPs by two different bacteria, Escherichia coli (EC) and Staphylococcus aureus (SA) and by their respective antibiotic-resistant phenotypes, MDR E. coli and MRSA, respectively, was performed. Samples of SeNPs are referred to herein using the following nomenclature: X-SeNPs, X being the short name of the bacterial strain used for the synthesis. After inoculation of the bacterial cultures with sodium selenite, a switch from yellowish to a red color was visible after 1 hour, and the color intensity increased until it reached a dark red color at 24 hours of synthesis. The change of color was due to the reduction of selenium ions presented in the media to elemental nanoparticles that could be either kept inside the bacterial cells or released to the media.

### Example 2: UV-Visible Analysis

Ultraviolet (UV) visible characterization (200-800 nm) was used to follow the progress of the synthesis of selenium nanoparticles (SeNPs) and the changes within the media in terms of nanoparticle production. Several aliquots were taken from the bacterial solution, once prior to inoculation with metallic salt, and then at several time points up to 24 hours. Aliquots were transferred to a 98-well plate and a full absorbance spectrum was recorded from 200 to 800 nm with 20 nm spacing. Different sodium selenite concentrations were employed for the inoculation with the aim to observe differences in bacterial behavior and reaction outcomes. Experiments were repeated three times, and the average of the measurements was calculated and plotted.

UV-visible spectroscopy showed the progression of the reaction for different concentrations of sodium selenite. Media and bacterial culture absorbance values were subtracted to only show the contribution of selenium to the reaction development. As can be seen, for a concentration of 2 mM sodium selenite for both E. coli (FIG. 1A) and S. aureus (FIG. 1B), the peak for selenium increased from 2 to 6 hours with a slight further increase for up to 24 hours. These results, which were observed for both bacterial strains, reveal that selenium production started shortly after the inoculation and continued up to 24 hours. FIG. 1A and FIG. 1B show absorbance versus wavelength for a concentration of 2 mM sodium selenite at 2, 6, 12, and 24 hours. No further increase was observed when the measurements were continued up to 48 hours (data not shown).

### Example 3: Kinetics Analysis

Bacterial suspensions inoculated with different concentrations of sodium selenite were prepared in a 96-well plate and introduced inside a SpectraMax M3 spectrophotometer machine for continuous measurements of absorbance following a kinetic study for a period of time up to 48 hours with measurements every 4 minutes. The experiments were done in triplicate, and the average data was converted from units of absorbance to CFU/mL using standardized calibration curves. Data were plotted and analyzed.

The kinetic study, completed for a time up to 48 hours, showed the bacterial response to the presence of different concentrations of sodium selenite. FIG. 2A and FIG. 2B show kinetics analyses (48 hours) for both E. coli and S. aureus, respectively. As can be seen, even the smallest concentration of the salt (1 mM) was able to cause a delay in the bacterial growth compared to the control. Specifically, in FIG. 2A, 1 mM delayed bacterial growth compared to control from 0 to about 15 hours. In FIG. 2B, 1 mM delayed bacterial growth compared to control from 0 to about 10 hours. Concentrations up to 5 mM prolonged this delay until a time around 10-12 hours, which is called the toleration point, at this moment, bacteria start growing as normal, following a proliferation curve similar to the one found in the control. On the other hand, 10 mM sodium selenite concentration was found to further delay the bacterial growth (see FIG. 2A), until a toleration point close to 25 hours, after which the bacteria started growing normally. For P. aeruginosa (plot not shown), all concentration of sodium selenite inhibited the normal bacterial growth, but the bacteria became tolerant after 24 hours.

### Example 4: Morphological Characterization of the Nanostructures

A morphological characterization of the nanostructures was done using transmission electron microscopy (TEM) (JEM-1010 TEM, JEOL USA Inc., MA). In order to prepare the samples for imaging, the nanoparticles were dried on 300-mesh copper-coated carbon grids (Electron Microscopy Sciences, Hatfield, Pa.). Additionally, an FEI Verios 460 Field Emission Microscope (FE-SEM) (FEI Europe B.V., Eindhoven, Netherlands) using selective secondary/backscattered electrons detection was also used for morphological characterization. The images were taken with 2 kV acceleration voltage and a 25 pA electron beam current. Electron dispersive (or energy-dispersive) X-Ray spectroscopy (EDX) was performed using an EDX detector (EDAX Octane Plus, Ametek B.V., Tilburg, Netherlands) coupled to the SEM previously mentioned, for the verification of the presence of elemental selenium in the structures. SEM conditions for EDX measurements were 10 kV acceleration voltage and 400 pA beam current.

Structural analysis of the nanostructures was carried out by infrared spectroscopy using a Fourier transform infrared spectrometer, Perkin Elmer 400 FT-IR/FT-NIR in attenuated total reflectance (ATR) mode. The samples for FT-IR analysis were prepared by drop casting the nanostructure colloids on a sample holder heated at around 50° C. IR spectra were scanned in the range of 500 to 4000 cm⁻¹ with a resolution of 4 cm⁻¹. The spectra were normalized, and the baseline corrected using Spectrum™ software from Perkin-Elmer.

Powder XRD patterns were obtained with a Rigaku MiniFlex 600 operating with a voltage of 40 kV, a current of 15 mA, and Cu—K_{α} radiation (λ=1.542 Å). All XRD patterns were recorded at room temperature with a step width of 0.05 (2Θ) and a scan speed of 0.25°/min. The preparation of the sample for XRD analysis was done by drying 2 mL of SeNPs colloids on the sample holder.

A SpectraMax M3 spectrophotometer (Molecular Devices, Sunnyvale, Calif.) was used to measure the optical density (OD) of the bacterial cultures. Growth curves and other bacterial analysis were performed in a plate reader SpectraMax® Paradigm® Multi-Mode Detection Platform.

For cell fixation studies, a Cressington 208HR High-Resolution Sputter Coater and a Samdri®-PVT-3D Critical Point dryer was used to prepare the samples, that were imaged using a Hitachi S-4800 SEM instrument was used with a 3 kV accelerating voltage and 10 μA of current.

### TEM Characterization

TEM images (FIGS. 3A-3D) show the nanostructures synthesized by MDR E. coli (FIG. 3A) and MRSA (FIG. 3B). After purification, the nanoparticles were completely removed from the bacterial cells and remained monodispersed, with a low degree of aggregation in solution, as can be seen for the SeNPs synthesized by both E. coli (FIG. 3C) and S. aureus (FIG. 3D).

Once the nanoparticles were released from the cells after purification, they were able to remain relatively monodispersed in solution due to the action of an organic coating that surrounded the nanospheres once they abandoned the bacterial matrix. This coating, whose analysis was accomplished later in this section, was made of biomolecules coming from the bacterial cells and was able to assemble to the outer layer of the SeNPs, as can be seen for nanoparticles made by both E. coli (FIG. 4A) and S. aureus (FIG. 4B). The SeNPs shown in FIGS. 4A and 4B were initiated with 2 mM of metallic salt concentration. The size of the nanospheres was measured using TEM measurements, plotting the average and analyzing the standard deviation, and the values can be seen in Table 1.

TABLE 1
Size distribution of SeNPs prepared by different bacterial strains

| Nanostructure | Diameter (nm) |
|---|---|
| MRSA-SeNPs | 62.5 ± 12.34 |
| SA-SeNPs | 72.1 ± 9.2 |
| MDR-SeNPs | 89.9 ± 20.2 |
| EC-SeNPs | 100.2 ± 31.2 |

### EDX Characterization

EDX characterization was performed on the four samples to confirm the presence of selenium within the samples. FIGS. 5A and 5B show the EDX spectra and quantification for EC-SeNPs (E. coli-SeNPs, FIG. 5A) and MDR-EC-SeNPs (FIG. 5B), with a high content of selenium, as well as carbon, oxygen, and nitrogen, indicating the presence of the organic coating surrounding the spheres.

For SA (S. aureus, FIG. 6A) and MRSA-SeNPs (FIG. 6B), the organic contribution of the spectra was found to be higher than the one in EC and MDR-EC-SeNPs. These results may be derived from the smaller size of the SeNPs synthesized by the Gram-positive (SA) bacteria that are able to form small aggregated embedded in organic matter.

### Cell Fixation for Synthesis

Cell fixation, combined with SEM microscopy, was employed to observe the process of synthesis of the nanoparticles once the synthetic protocol was completed (24 hours after inoculation of the metallic salt to the bacterial population). As can be seen, MDR E. coli bacteria (FIGS. 7A and 7B) were able to generate relatively monodispersed and uniformly sized selenium nanospheres that were spread all over the surface of the cells, with no apparent disruption of the cellular membranes. On the other hand, MRSA bacteria (FIGS. 7C and 7D) appeared to form smaller nanoparticles that were present all over the surface of the membranes, with some level of aggregation observed in the space between cells.

### XRD Characterization

The X-ray diffraction (XRD) patterns for EC-SeNPs, SA-SeNPs, MDR-EC-SeNPs, and MRSA-SeNPs are shown and compared in FIG. 8. Some amorphous hump or halo is visible in the top three XRD spectra shown in FIG. 8. The diffraction peaks for the MRSA-SeNPs XRD pattern (bottom spectrum, FIG. 8) may be indexed to trigonal Se structure (α-Se, space group P3121). The XRD analysis indicated the lack of presence of lower intensity peaks, probably due to the very low signal to noise ratio of the samples.

In the case of the XRD analysis of EC-SeNPs, SA-SeNPs, and MDR-EC-SeNPs, (see FIG. 8), the XRD patterns indicate a more abundant amorphous phase than in sample MRSA-SeNPs. However, in the samples EC-SeNPs and SA-SeNPs, it can be noted that the most intense peak is located at diffraction of 2Θ=31°, which is closely related to trigonal selenium.

### FTIR Characterization

The FT-IR spectra of samples EC-, MDR EC-, SA- and MRSA-SeNPs are shown in FIG. 9. In general, the samples showed a broad signal around 3270 cm⁻¹ that is characteristic of the OH bond and a very weak asymmetrical stretching band centered at 2960 cm⁻¹ that is representative of CH₃. At the wavenumber 2925 cm⁻¹, there is an asymmetrical vibration due to CH₂ that is found in proteins Around 1625, 1520, and 1234 cm⁻¹, further protein vibrational stretching signals are found that represent amide I, II, III bonds. Carboxylate (COO—) signals related to amino acids can be located at 1452 and around 1390 cm-1, these bands correspond to the bending and symmetrical vibrations of the COO— ion. The vibrational band localized at 1313 cm⁻¹ may be related to the C—H deformation signal that normally occurs in proteins. All of the samples, excluding MDR-EC-SeNPs, contain a signal at 1157 cm⁻¹ common on proteins with CH₂ wagging vibration. Finally, all SeNPs present a band around 1070 cm⁻¹ that is characteristic of stretching vibrations in CO bonds. According to the overall assignment of the vibrational signals found in all the FT-IR spectra, there might be a correlation of threonine related proteins being functionalized more frequently in the Se-based NPs. Additional peak assignation can be found in Table 2.

TABLE 2
Peak assignation of FTIR analysis.

| Wave- | | | | |
|---|---|---|---|---|
| number/ | | | Type of | |
| cm⁻¹ | Assignment | Type of band | molecule | Reference |
| 3270 | OH | Stretching vibration | Amino | Kora2017 |
| | | | acids | |
| 2960 | CH in CH₃ | Vibration asymmetrical | Proteins | Kamne v2017 |
| | | Stretching | | |
| 2920 | CH in CH₂ | Vibration asymmetrical | Proteins | Kamne v2017 |
| | | Stretching | | |
| 1622 | NH amide I | Vibration stretching | Proteins | Kora2017 |
| 1531 | NH amide II | Vibration stretching | Proteins | Kora2017 |
| 1452 | CH₃ + COO | Bending vib. + vib. | Proteins | Kamne v2017 |
| | | Asymmetrical | | |
| 1395 | COO | Vibration sym. | Amino | Kamne v2017 |
| | | | acids | |
| 1340 | Amide III | Vibration sym. | Proteins | Kamne v2017 |
| 1313 | CH | Deformation | Proteins | Barth2007 |
| 1234 | Amide III | Vibration Asymmetrical | Proteins | Kamne v2017 |
| 1158 | CH₂ | Wagging vibration | Proteins | Barth2007 |

### Example 5: Stability Analysis

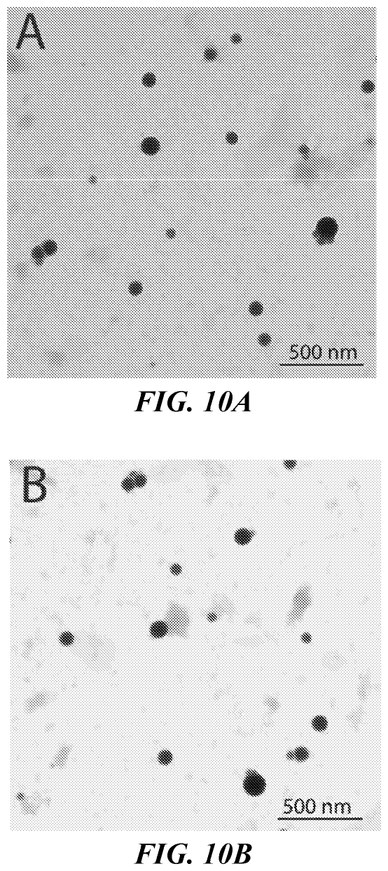

In order to analyze the stability of the samples, TEM and Zeta-potential measurements were carried out using fresh and 60-days old nanoparticles (TEM images in FIGS. 10A and 10B). In general, it was evident that the samples kept their original morphologies and features. For example, the 60-days old SA-SeNPs (FIG. 10A) sample showed nanoparticles that remained monodispersed in solution, together with some isolate aggregation cases, with small nanospheres agglomerated with bigger ones. A similar result was observed for EC-SeNPs (FIG. 10B) that remained monodispersed in solution. The SeNPs shown in FIGS. 10A and 10B were grown with 2 mM metallic salt concentration. These features are in accordance with the freshly synthesized nanomaterials, as can be seen for comparison in the TEM images in FIG. 3C and FIG. 3D.

The stability analysis was carried out through the measurement of the Z-potential of the freshly synthesized and 60-days old Se-based nanomaterials. In general, a colloid or suspension is considered stable if the Z-potential is above a critical value of ±30 mV. Given the measured Z-potential values for the colloids (freshly and 60-days old samples, see Table 3), they can be considered highly stable, what is in accordance with the TEM images.

TABLE 3
Zeta-potential values for freshly and 60-days old MDR-, EC-,
MRSA-, SA-SeNPs

| | Z-potential (mV) | |
|---|---|---|
| Nanostructures | As-synthesized | 60 days old |
| MDR-SeNPs | −65.03 ± 9.65 | −60.12 ± 4.51 |
| EC-SeNPs | −72.6 ± 3.03 | −72.34 ± 2.24 |
| MRSA-SeNPs | −69.88 ± 3.30 | −67.26 ± 1.10 |
| SA-SeNPs | −66.67 ± 4.45 | −65.11 ± 4.55 |

The nanoparticles were unlikely to form aggregates as a consequence of their electrostatic stability. Neutral and negatively charged NPs tend to have long half-lives in human serum and are not taken up by cells in a non-specific manner (Alexis et al., 2008).

### Example 6: Testing the Antimicrobial Effect of the Nanostructures

A colony of each bacterial strain was re-suspended in LB media. The bacterial suspension was placed in a shaking incubator to grow overnight at 200 rpm and 37° C. The overnight suspension was diluted to a bacterial concentration of 10⁶ colony forming units per milliliter (CFU/mL), and a spectrophotometer was used to perform optical density measurements at 600 nm (OD600). The colony counting assays were done by seeding the bacteria in the wells of a 96-well plate and mixed with different concentrations of various biosynthesized-SeNPs. The plates were incubated at 37° C. for 8 hours. Next, the plates were removed from the incubator and diluted with PBS in a series of vials 10⁵-fold, 10⁶-fold and 10⁷-fold. Three 10 μL drops were taken of each dilution and deposited in an LB-Agar plate. After a final period of incubation of 8 hours inside the incubator at 37° C., the numbers of colonies formed were counted.

Colony counting unit assays were conducted to assess the potential antimicrobial activity of the different bacterial-synthesized SeNPs. An extensive study was conducted, divided into two main categories: straight analysis, in which a nanoparticle made by X bacteria—for instance, E. coli—was tested towards the X bacterial strain, both antibiotic-resistant and standard phenotypes, for instance, E. coli and MDR E. coli; and crossed analysis, in which a nanoparticles made by X bacteria—for instance, E. coli—were tested towards the Y bacterial strain, both antibiotic-resistant and standard phenotypes—for instance, S. aureus and MRSA.

The straight analysis for E. coli (FIGS. 11A and 11B) and for MDR E. coli (FIGS. 11C and 11D) showed a clear dose-dependent inhibition of the bacteria when they were cultured with EC-SeNPs and with MDR-SeNPs. The picture showed a promising antibacterial effect in a range of EC-SeNPs concentrations between 25 to 100 μg/mL due to a decrease and inhibition in bacterial growth for E. coli. For MDR-SeNPs, the concentrations between 25 to 100 μg/mL showed the antibacterial effects for MDR E. coli bacteria. To summarize FIGS. 11A-11D, the colony counting assay of E. coli and MDR-E. coli after being treated for 8 hours with different bacteria-mediated synthesized nanoparticles are shown; and the data=mean+/−SEM, N=3, *p<0.05 versus control (0 μg/mL concentration), **p<0.01 versus control (0 μg/mL concentration).

The straight analysis for MRSA-SeNPs and SA-SeNPs showed a clear dose-dependent inhibition of S. aureus when cultured with SA-SeNPs in FIG. 12A and with MRSA-SeNPs in FIGS. 12B and 12C, respectively. The analysis of data showed a promising antibacterial effect in a range of MRSA-SeNPs concentrations between 50 to 100 μg/mL due to a decrease and inhibition in bacterial growth for S. aureus. For MRSA-SeNPs, the concentrations between 25 to 100 μg/mL showed the antibacterial effects for MRSA (FIG. 12C). However, no inhibition was found when the SA-SeNPs were tested towards the antibiotic-resistant phenotype in FIG. 12D. FIGS. 12A-12D show colony counting assays of S. aureus and MRSA after being treated for 8 hours with different bacteria-mediated synthesized nanoparticles. The data=mean+1-SEM, N=3, *p<0.05 versus control (0 μg/mL concentration), **p<0.01 versus control (0 μg/mL concentration).

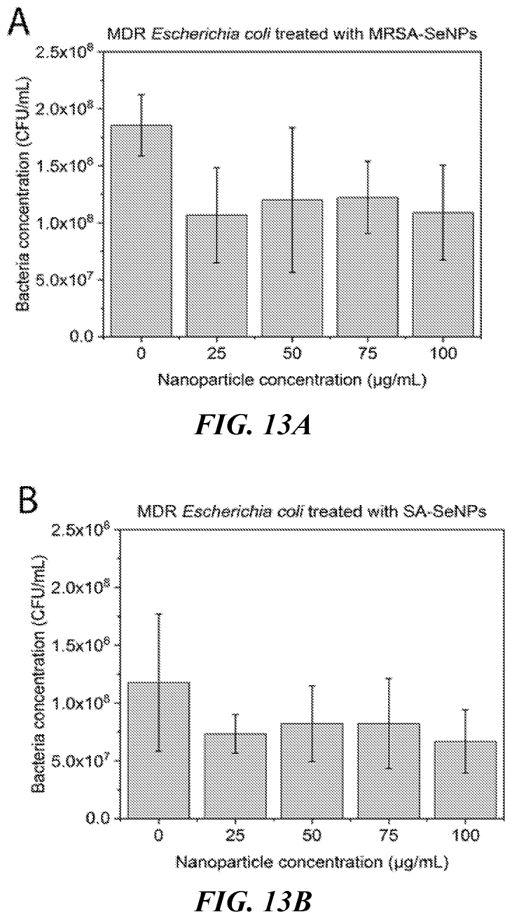

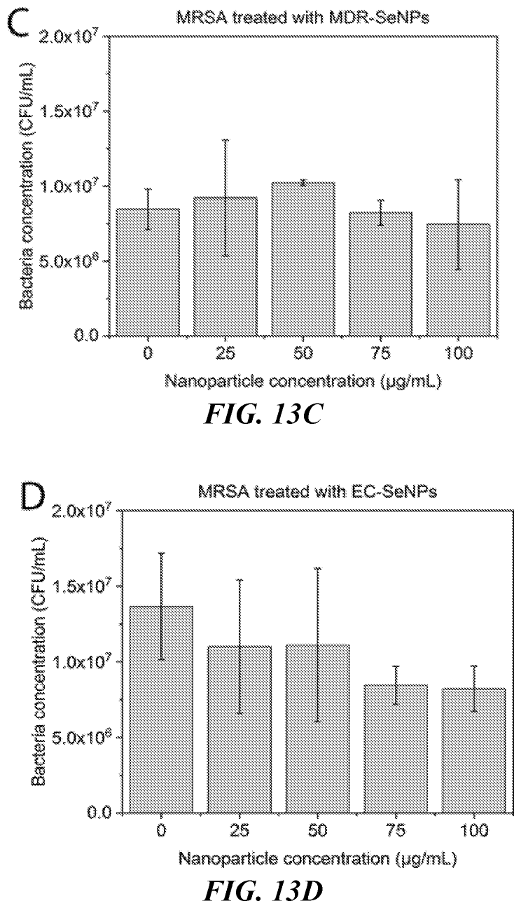

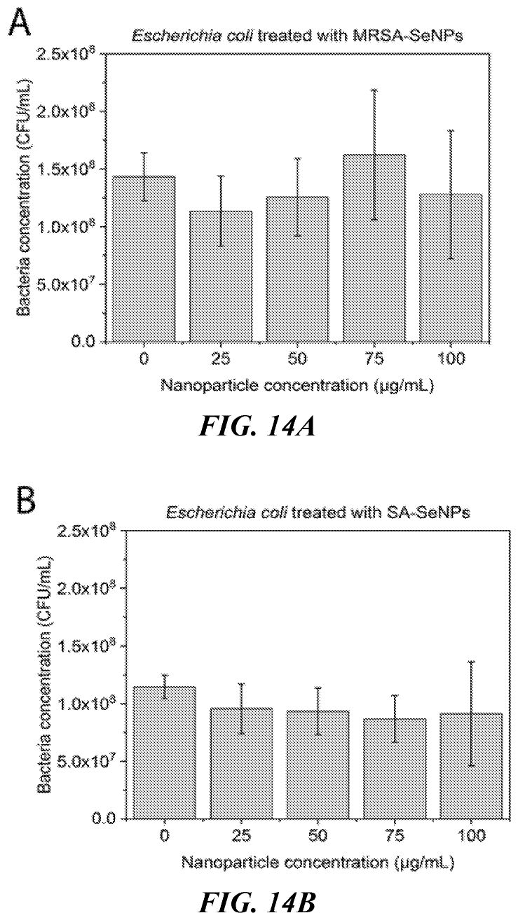

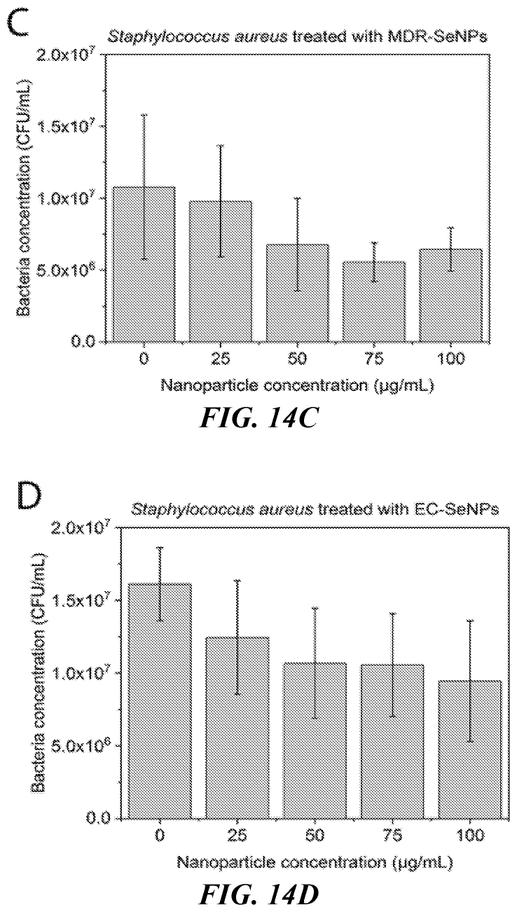

The crossed analysis for MDR E. coli and MRSA treated with MRSA-SeNPs, SA-SeNPs, MDR-SeNPs, and EC-SeNPs (FIGS. 13A-13D), show no inhibition was found when the nanoparticles were tested towards the standard and antibiotic-resistant phenotypes. To summarize FIGS. 13A-13D, colony counting assays of MDR-E. coli and MRSA after being treated for 8 hours with different bacteria-mediated synthesized nanoparticles (MRSA-SeNPs, SA-SeNPs, MDR-EC-SeNPs, and EC-SeNPs) are shown; the data=mean+/−SEM, N=3, *p<0.05 versus control (0 μg/mL concentration), **p<0.01 versus control (0 μg/mL concentration). The crossed analysis for E. coli and S. aureus treated with MRSA-SeNPs, SA-NPs, MDR-SeNPs, and EC-SeNPs (FIGS. 14A-14D) showed no inhibition when the nanoparticles were tested towards the standard and antibiotic-resistant phenotypes. FIGS. 14A-14D show colony counting assay of E. coli and S. aureus after being treated for 8 hours with different bacteria-mediated synthesized nanoparticles (MRSA-SeNPs, SA-SeNPs, MDR-EC-SeNPs and EC-NPs); and the data=mean+/−SEM, N=3, *p<0.05 versus control (0 μg/mL concentration), **p<0.01 versus control (0 μg/mL concentration).

The minimum inhibitory concentrations (MIC) was calculated (Table 4) to quantify further the antibacterial effect of the nanoparticles for those experiments that showed the antibacterial effect.

TABLE 4
MIC values for different nanoparticles against different bacteria.

| | EC- | MDR- | SA- | MRSA- |
|---|---|---|---|---|
| Experiment | SeNPs + EC | SeNPs-MDR | SeNPs-SA | SeNPs + MRSA |
| MIC (μg/mL) | 30.03 | 28.82 | 26.35 | 19.22 |

These values differ from others found in literature, showing either a decrease or similitudes of the MIC values for the four nanosystems. For example, Srivastava et al. showed that bacteria-mediated SeNPs produced by the R. eutropha biomass, tested towards S. aureus and E. coli, rendered a MIC value of 100 μg/mL, while Hariharan et al. reported the MIC of microbially-synthesized SeNPs towards E. coli and S. aureus, with values close to 30 μg/mL.

### Example 7: Testing the Effect of the Nanomaterials Towards Human Cells

Cytotoxicity assays were performed with primary human dermal fibroblasts (ATCC® PCS-201-012TM, Manassas, Va.)) and melanoma (ATCC® CRL-1619, Manassas, Va.) cells. Cells were cultured in Dulbecco's Modified Eagle Medium (DMEM; Thermo Fisher Scientific, Waltham, Mass.), supplemented with 10% fetal bovine serum (FBS; ATCC® 30-2020™, American Type Culture Collection, Manassas, Va.) and 1% penicillin/streptomycin (Thermo Fisher Scientific, Waltham, Mass.). MTS assays (CellTiter 96® AQueous One Solution Cell Proliferation Assay, Promega, Madison, Wis.) were carried out to assess cytotoxicity. Cells were seeded onto tissue-culture-treated 96-well plates (Thermo Fisher Scientific, Waltham, Mass.) at a final concentration of 5000 cells per well in 100 μL of cell medium. After an incubation period of 24 hours at 37° C. in a humidified incubator with 5% carbon dioxide (CO₂), the culture medium was replaced with 100 μL of fresh cell medium containing concentrations from 25 to 100 μg/mL of bacterial-synthesized SeNPs. Cells were cultured for another 24 and 48 hours at the same conditions and then washed with PBS, the medium was then replaced with 100 μL of the MTS solution (prepared using a mixing ratio of 1:5 of MTS:Medium). After the addition of the solution, the 96-well plate was incubated for 4 hours in the incubator to allow for a color change. Then, the absorbance was measured at 490 nm on an absorbance plate reader (SpectraMAX M3, Molecular Devices) for cell viability after exposure to the bio-SeNPs' concentration. Cell viability was calculated by dividing the average absorbance obtained for each sample by the one achieved by the control sample and then multiplied by 100. Controls containing cells and media, and just media, were also included in the 96-well plate to identify the normal growth of cells without nanoparticles and to determine the absorbance of the media itself.

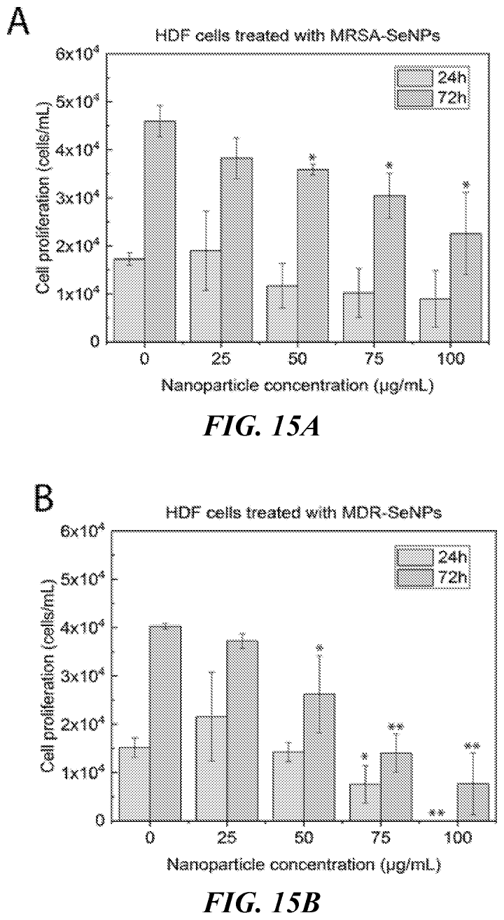

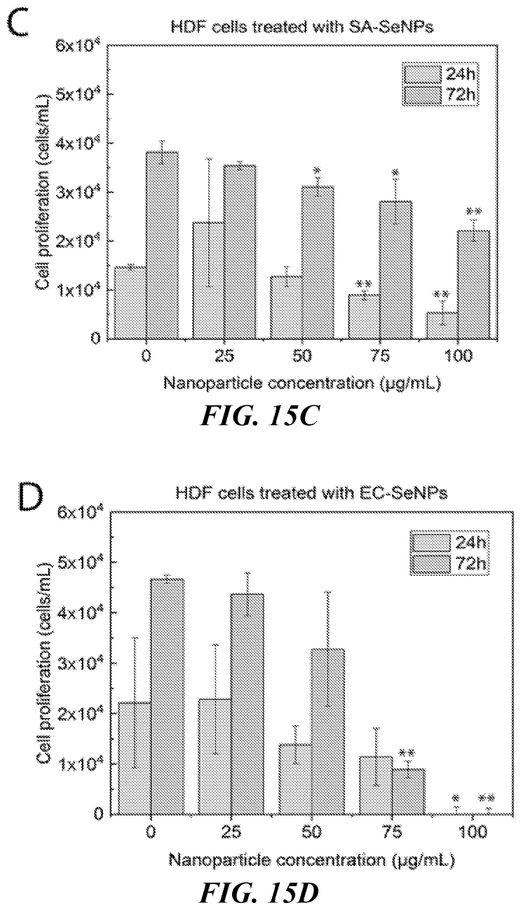

With the aim to determine the cytotoxicity associated with the bacteria-synthesized SeNPs in mammalian cells, in vitro cytotoxicity assays were performed with HDF and human melanoma cells for 24 hours and 72 hours. A dose-dependent cell proliferation decay was found when the four nanostructures were cultured with HDF cells over a period of time of 3 days (FIGS. 15A-15D). Experiments with fibroblast cells showed a decrease in the cell viability with a nanoparticle concentration increase for all the systems, with a constant cell proliferation after 72 hours of growth. For MRSA-SeNPs (FIG. 15A), a low cytotoxic effect was found in a range of concentrations between 25 to 100 μg/mL at 24 hours, while the range was reduced at concentrations up to 50 μg/mL at the third day. Besides, an important depletion of the cell proliferation was found at concentrations higher than 50 μg/mL and 75 μg/mL for MDR-SeNPs (FIG. 15B), SA-SeNPs (FIG. 15C) and EC-SeNPs (FIG. 15D) after 72 hours of exposure. FIGS. 15A-15D show MTS assays on human dermal fibroblast (HDF) in the presence of MRSA-SeNPs (A), MDR-SeNPs (B), SA-SeNPs (C) and EC-SeNPs (D) ranging from 25 to 100 μg/mL; and the data=mean+/−SEM, N=3, *p<0.05 versus control (0 μg/mL concentration), **p<0.01 versus control (0 μg/mL concentration).

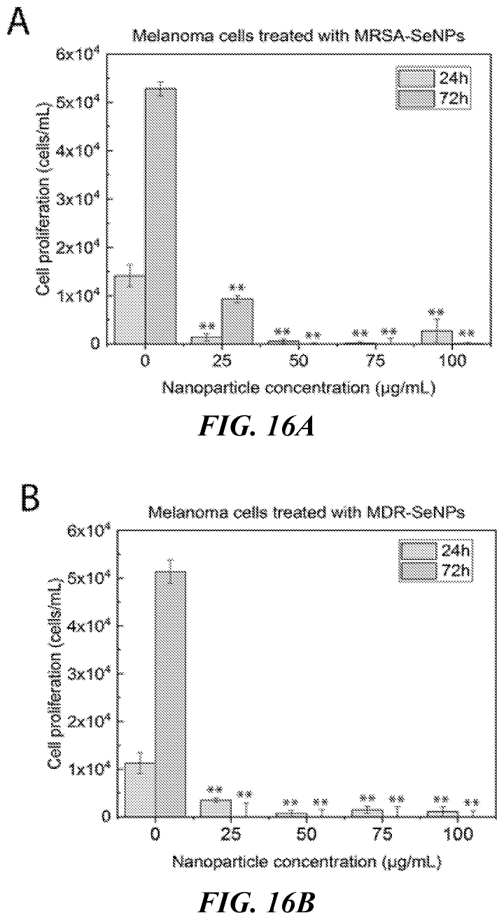

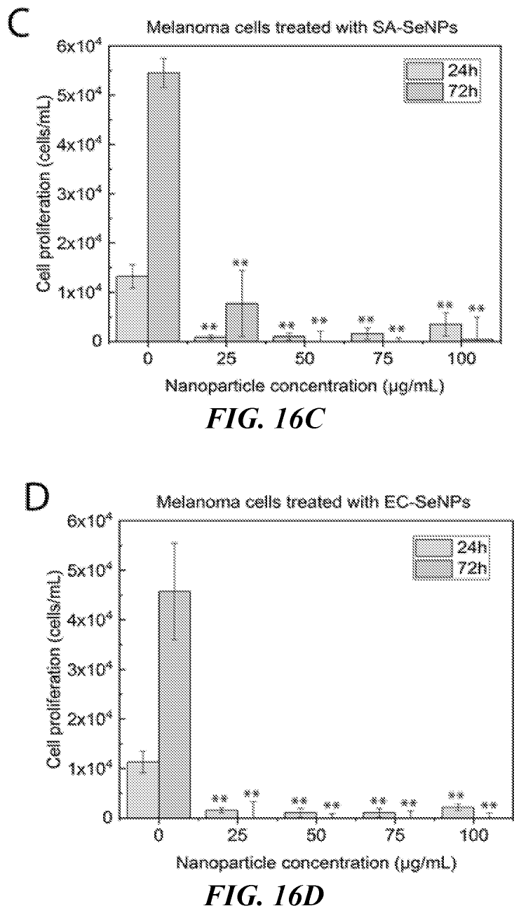

The second set of experiments was carried out melanoma cells that were used to evaluate the potential anticancer effect of the SeNPs (FIGS. 16A-16D). A dose-dependent cell proliferation decay was found when the bacteria-mediated were cultured with melanoma cells for 1 day and 3 days. MRSA-SeNPs (FIG. 16A) showed inhibition of cell growth in the extended range of concentrations. Nevertheless, it was accentuated at concentrations higher than 25 μg/mL. With similar behavior, MDR-SeNPs (FIG. 16B), SA-SeNPs (FIG. 16C) and EC-SeNPs (FIG. 16D) presented a remarkable decrease in cell proliferation. Since concentrations of 25 μg/mL showed an important decrease of cell proliferation for all the systems, it was confirmed that a low density of metallic nanoparticles can trigger a significant anticancer activity, with low cytotoxicity towards healthy human cells. FIGS. 16A-16D show MTS assay on human melanoma cells in the presence of MRSA-SeNPs (A), MDR-SeNPs (B), SA-SeNPs (C) and EC-SeNPs (D) ranging from 25 to 100 μg/mL; and the data=mean+/−SEM, N=3, *p<0.05 versus control (0 μg/mL concentration), **p<0.01 versus control (0 μg/mL concentration).

IC₅₀ values were calculated to further study the response of the cells to the nanostructures (Table 5).

TABLE 5
IC₅₀ values for different nanoparticles cultured with melanoma cells.

| IC₅₀ (μg/mL) | 1 day | 3 days |
|---|---|---|
| MRSA-SeNPs | 20.10 | 15.04 |
| SA-SeNPs | 12.04 | 10.73 |
| MDR-SeNPs | 8.23 | 9.10 |
| E-SeNPs | 7.60 | 14.88 |

These values differed from others found in literature, showing a decrease of the IC₅₀ values for our nanosystems. For example, Vekariya et al. have investigated the anticancer effect of green synthesized SeNPs. The nanoparticles were tested against early-stage breast cancer cell line (MCF-7), with an IC₅₀ value of 25 μg/ml for a 1-day treatment. Moreover, T. Chen et al. synthesized SeNPs that were tested against A375 melanoma cell lines, rendering IC₅₀ values greater than 17.6 μg/mL, after 24 hours of treatment.

### Example 8: Cell Fixation and SEM Imaging

For the fixation of bacterial cells, both bacterial strains (MDR E. coli, E. coli, S. aureus and MRSA) were inoculated into 4 mL of sterile LB media in a 15 mL Falcon conical centrifuge tube and incubated at 37° C. at 200 rpm for 24 hours. The optical density was then measured at 600 nm (OD600) using a spectrophotometer. The overnight suspension was diluted to a final bacterial concentration of 10⁶ colony forming units per milliliter (CFU/mL) prior to measuring the optical density. A selected 75 μg/mL concentration of MRSA-SeNPs, MDR-SeNPs, SA-SeNPs, and EC-SeNPs was mixed with LB media and bacterial solution in a 6-well plate with a glass coverslip attached to the bottom. The coverslips were pre-treated with poly-lysine to enhance cell adhesion right before the experiment. The plate was placed inside an incubator for 8 hours at 37° C. After the experiments, the coverslips were fixed with a primary fixative solution containing 2.5% glutaraldehyde and 0.1 M sodium cacodylate buffer solution for 1 hour. Subsequently, the fixative solution was exchanged for 0.1 M sodium cacodylate buffer and the coverslips were washed 3 times for 10 min. Post-fixation was done using 1% osmium tetroxide (OsO4) solution in the buffer for 1 hour. Subsequently, the coverslips were washed three times with buffer and dehydration was progressively achieved with 30, 50, 70, 80, 95 and 100% ethanol (three times for the 100% ethanol). Finally, the coverslips were dried by liquid CO₂-ethanol exchange in a Samdri®-PVT-3D Critical Point Dryer. The coverslips were mounted on SEM stubs with carbon adhesive tabs (Electron Microscopy Sciences, EMS) after treatment with liquid graphite, and then sputter coated with a thin layer of platinum using a Cressington 208HR High-Resolution Sputter Coater. Digital images of the treated and untreated bacteria were acquired using an SEM.

SEM micrographs of control MDR E. coli and MRSA (FIGS. 17A and 17C, respectively) and bacteria after treatment with MDR-SeNPs (FIG. 17B) and MRSA-SeNPs (FIG. 17D) were taken to further analyze the effect of the nanoparticles within the bacterial media. The characterization indicated that the treatment with the bacteriogenic SeNPs induced a change of both bacterial strains. Disruption of the outer cell membrane and cell lysis were seen after the treatment. Therefore, clear cell damage was observed, with an abundant presence of holes and cracks all over the cell membrane, as well as bacterial deformation and collapse. The cell membrane damage is commonly found to be a cause of ROS. Nevertheless, other mechanisms can also be inferred, as the direct damage of the cells due to the morphology of the nanostructures. From the SEM images of the bacteria, it is possible to see that the membrane damage occurs and that there was the attachment of nanoparticles to bacteria, but the exact mechanism how damage occurs could not be identified. FIGS. 17A-17D show SEM micrographs of control MDR E. coli and MRSA (A, C) and bacteria after treatment with MDR-SeNPs and MRSA-SeNPs (B, D), respectively.

### Example 9: Reactive Oxygen Species (ROS) Analysis of Samples

For ROS quantification, 2′,7′-dichlorodihydrofluorescein diacetate (H₂DCFDA) was used. Human melanoma cells were seed in a 96 well-plate at a concentration of 5×10⁴ cells/mL in the presence of different concentrations of the SeNPs as well as in control without any nanoparticles. The cells were cultured under standard culture conditions (37° C. in a humidified incubator with a 5% carbon dioxide (CO₂) atmosphere) for 24 h before the experiment. Briefly, the ROS indicator was reconstituted in anhydrous dimethylsulfoxide (DMSO) to make a concentrated stock solution that was kept and sealed. The growth media were then carefully removed, and a fixed volume of the indicator in PBS was added to each one of the wells at a final concentration of 10 μM. The cells were incubated for 30 min as optimal temperature, and the loading buffer was removed after. Fresh media were added, and cells were allowed to recover for a short time. The baseline for fluorescence intensity of a sample of the loaded cell period exposure was determined. Positive controls were done stimulating the oxidative activity with hydrogen peroxide to a final concentration of 50 μM. The intensity of fluorescence was then observed by flow cytometry. Measurements were taken by an increase in fluorescence at 530 nm when the sample was excited at 485 nm. Fluorescence was also determined in the negative control, untreated, loaded with dye cells maintained in a buffer.

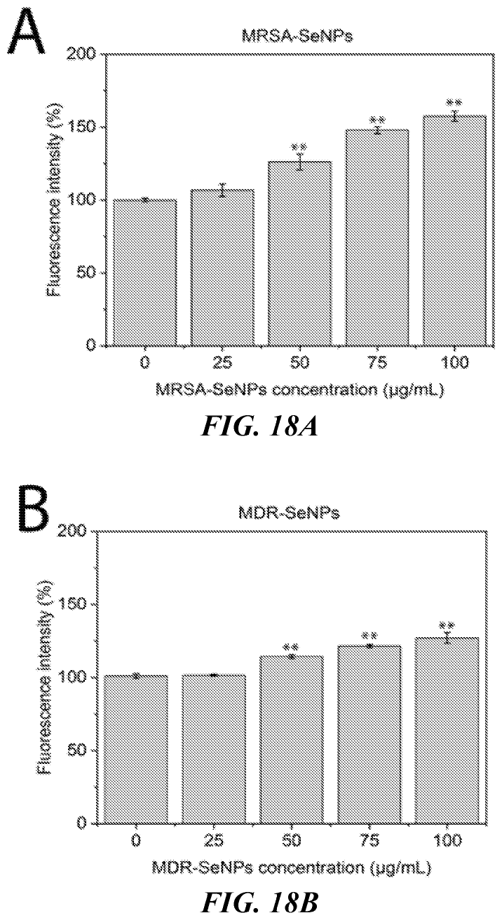

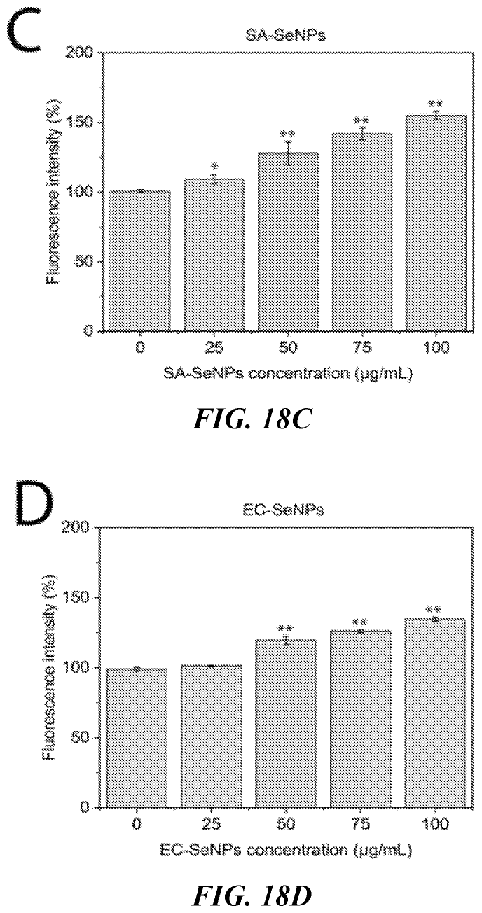

ROS analysis (FIG. 18) showed an increase in ROS production when nanoparticles were present in the media, with a dose-dependent effect. Therefore, the increase in the number of reactive oxygen species was related to the dose-dependent anticancer behavior that was shown before. FIGS. 18A-18D show the results of ROS study of MRSA-SeNPs analysis (FIG. 18A), MDR-SeNPs analysis (FIG. 18B), SA-SeNPs (FIG. 18C) and EC-SeNPs (FIG. 18D).

1. A method of inhibiting the growth of a drug-resistant bacterial pathogen in a subject, the method comprising administering selenium nanoparticles to the subject, whereby the growth of the bacterial pathogen in the subject is inhibited;
wherein the selenium nanoparticles are produced by a process comprising growing the bacterial pathogen in the presence of a selenium salt, whereby selenium ions of the selenium salt are reduced to elemental selenium to form the selenium nanoparticles; and
wherein the selenium nanoparticles selectively inhibit growth of the drug-resistant bacterial pathogen compared to inhibition by the selenium nanoparticles of growth of a non-drug-resistant form of the bacterial pathogen.

2. The method of claim 1, wherein the selenium nanoparticles are at least partially coated with organic molecules provided by the bacterial pathogen during the process of producing the selenium nanoparticles.

3. The method of claim 1, wherein the drug-resistant bacterial pathogen is of the same species as the non-drug-resistant form of the bacterial pathogen.

4. The method of claim 1, wherein both the drug-resistant and non-drug-resistant forms of the bacterial pathogen are Escherichia coli, or both the drug-resistant and non-drug-resistant forms are Staphylococcus aureus.

5. The method of claim 1, wherein a minimum inhibitory concentration of the selenium nanoparticles for the drug-resistant bacterial pathogen is less than about 30 micrograms/mL.

6. The method of claim 1, further comprising, prior to said administering:
collecting a sample of the drug-resistant bacterial pathogen from the subject;
cultivating the collected drug-resistant bacterial pathogen in vitro; and
forming said selenium nanoparticles by growing the cultivated bacterial pathogen in the presence of said selenium salt, whereby selenium ions of the selenium salt are reduced to elemental selenium to form said selenium nanoparticles.

7. The method of claim 1, wherein the administered selenium nanoparticles are formulated with one or more pharmaceutically acceptable excipients.

8. The method of claim 1, wherein the administered selenium nanoparticles comprise one or more radioisotopes, and the method further comprises performing radioimaging of the subject, irradiation of the subject by the selenium nanoparticles, or absorption of radiation from the selenium nanoparticles by elemental selenium in the nanoparticles and emission of energy from the selenium nanoparticles.

9. The method of claim 1, wherein the selenium nanoparticles possess magnetic properties operative to collect, concentrate, organize, dissipate, or repel the nanoparticles.

10. The method of claim 1, wherein the selenium nanoparticles comprise a moiety selected from the group consisting of a protein, an antibody, an oligonucleotide, and a small molecule drug.

11. The method of claim 10, wherein the moiety is a targeting moiety capable of targeting the selenium nanoparticles to the drug-resistant bacterial pathogen or to a cell of the subject.

12. The method of claim 1, wherein the selenium nanoparticles cause a lethal increase in reactive oxygen species in the drug resistant bacteria.

13. A method of inhibiting the growth of cancer cells, the method comprising administering to a subject in need thereof a therapeutically effective amount of selenium nanoparticles; wherein the selenium nanoparticles are produced by a process comprising growing bacteria in the presence of a selenium salt wherein selenium ions of the salt are reduced to elemental selenium to form the nanoparticles.

14. The method of claim 13, wherein the cancer cells are cells of a cancer selected from the group consisting of skin cancer, lung cancer, breast cancer, prostate cancer, colorectal cancer, bladder cancer, melanoma, Non-Hodgkin lymphoma, kidney cancer, and leukemia.

15. The method of claim 13, wherein the growth of non-cancerous cells in the subject is not substantially inhibited.

16. The method of claim 15, wherein the therapeutically effective amount provides a concentration of selenium nanoparticles not greater than about 25 micrograms/mL at or near the cancer cells.

17. The method of claim 13, wherein the selenium nanoparticles cause a lethal increase in reactive oxygen species in the cancer cells.

18. Selenium nanoparticles produced by a process comprising growing a first type of bacteria in the presence of a selenium salt, wherein selenium ions of the salt are reduced to elemental selenium, wherein the selenium nanoparticles selectively inhibit growth of the first type of bacteria more than the selenium nanoparticles inhibit growth of a second type of bacteria.

19. The selenium nanoparticles of claim 18, wherein the selenium nanoparticles are at least partially coated with organic molecules provided by the bacterial pathogen during the process of producing the selenium nanoparticle.

20. The selenium nanoparticles of claim 19, wherein the organic coating causes the selenium nanoparticles to selectively inhibit growth of the first type of bacteria compared to other types of bacteria.

21. The selenium nanoparticles of claim 19, wherein the organic coating comprises one or more proteins.

22. The selenium nanoparticles of claim 18, wherein the selenium nanoparticles further comprise a moiety selected from the group consisting of a radioisotope, a protein, an antibody, an oligonucleotide, a small molecule, and a therapeutic agent.

23. The selenium nanoparticles of claim 18, wherein the first type of bacteria is drug-resistant.

24. The selenium nanoparticles of claim 23, wherein the drug resistance is antibiotic resistance.

25. The selenium nanoparticles of claim 18, wherein the first bacteria are multi-drug resistant Escherichia coli or methicillin-resistant Staphylococcus aureus.

26. The selenium nanoparticles of claim 18, wherein the selenium nanoparticles comprise amorphous selenium and/or trigonal selenium crystal structure.

27. The selenium nanoparticles of claim 18, wherein the nanoparticles have an average diameter in the range from about 50 nm to about 110 nm, or about 50 to about 75 nm, or about 70 nm to about 110 nm.

28. The selenium nanoparticles of claim 18, wherein the organic coating is operative to stabilize the selenium nanoparticles as a colloid or suspension for at least about 60 days.

29. The selenium nanoparticles of claim 18, wherein the organic coating provides a Z-potential value exceeding ±30 mV which is stable for at least about 60 days.

30. The selenium nanoparticles of claim 18, wherein the first type of bacteria is a drug-resistant form of the second type of bacteria.

31. The selenium nanoparticles of claim 18 that are capable of inhibiting proliferation of cancer cells without significantly inhibiting proliferation of non-cancer cells of a human subject.

32. The selenium nanoparticles of claim 31, wherein the cancer cells are melanoma cells and the normal cells are dermal fibroblasts.

33. A pharmaceutical composition comprising the selenium nanoparticles of claim 18 and a pharmaceutically acceptable excipient.

34. A kit for inhibiting the growth of drug-resistant bacteria, the kit comprising
a selenium salt; and
instructions for carrying out the method of claim 6.
